(19) United States
(12) Patent Application Publication
(10) Pub. No.: US 2025/0306022 A1
(43) Pub. Date: Oct. 2, 2025

(54) METHOD FOR CLASSIFYING CELLS

(71) Applicant: Hoffmann-La Roche Inc., Little Falls, NJ (US)

(72) Inventors: Katharina Alice Maria Essig, München (DE); Elke GLASMACHER, Muenchen (DE); Carsten MARR, München (DE); Fabian SCHMICH, Bad Feilnbach (DE); Sayedali Shetab Boushehri, München (DE)

(73) Assignee: Hoffmann-La Roche Inc., Little Falls, NJ (US)

(21) Appl. No.: 19/177,167

(22) Filed: Apr. 11, 2025

Related U.S. Application Data
(63) Continuation of application No. PCT/EP2023/077950, filed on Oct. 10, 2023.

(30) Foreign Application Priority Data
Oct. 12, 2022 (EP) 22201067.0

(51) Int. Cl. G01N 33/569; G01N 33/68

(52) U.S. Cl. CPC G01N 33/56972; G01N 33/56977; G01N 33/6863; G01N 2333/7051; G01N 2333/70539; G01N 2333/70596

(57) ABSTRACT

Herein is reported a method for classifying cells in a cell mixture, wherein the mixture comprises T-cells and B-cells, comprising the steps of first applying at least labelled antibodies binding to F-actin, MHCII and CD3 to the cell mixture to obtained a labelled cell mixture, wherein the antibodies are each labelled with a dye, wherein the dyes have different (non-overlapping) emission wavelengths, second acquiring at least one image of the cell mixture, and third classifying the cells in the cell mixture to be an isolated cell if the cell is a single cell, is F-Actin positive and is MHCII positive and CD3 negative, or is MHCII negative and CD3 positive, or to be a doublet or multiplet of cells if the cell is an aggregate of two or three cells, is F-Actin positive, MHCII positive and CD3 positive.

The current invention is in the field of analytical technologies. In more detail, herein is reported a method for classifying cells in a mixture of B- and T-cells based on differential labelling into isolated cells, cell multiplets without signaling and cell multiplets with signaling. Such a classification allows for the characterization of therapeutics interfering with the formation of cell signaling

### BACKGROUND OF THE INVENTION

Therapeutic antibodies are widely used to treat severe diseases. Most of them alter immune cells and act within the immunological synapse: an essential cell-to-cell interaction to direct the humoral immune response. Although many antibody designs are generated and evaluated, a high-throughput tool for systematic antibody characterization and prediction of function is lacking.

The formation of an immunological synapse is the first event of the adaptive immune reaction induced by the interaction of a T-cell with its corresponding antigen-presenting cell (APC). This rapidly formed cell-cell interface is initiated by the recognition of peptide-loaded MHC complexes by the T-cell receptor (TCR). It involves the rearrangement of actin filaments of the cytoskeleton and the recruitment of signaling, co-stimulatory, co-inhibitory, and adhesion molecules to the nascent synapse [1,2]. This process is crucial to trigger and fine-tune T-cell responses and ensure intact immune reactions. Dysfunctional immunological synapse formation has been observed in several immune-related disorders [3-8] and has thus been considered a potential target to stimulate or inhibit immune responses by modulating its assembly or function [9-11]. For instance, various therapeutic antibodies were developed that alter immunological synapse formation to treat cancer and autoimmune diseases [12-15]. Although significant progress in developing immunological synapse targeting agents has been achieved in the last years [9], there is still need to refine the compounds further, especially to improve their efficacy. It has been identified that antibody size and format [16,17], the dose, as well as target expression [18] can be critical parameters for immunological synapse formation and its effect on T-cell function.

However, so no method to systematically quantify and characterize the morphology of the immunological synapse, investigate its correlation to T-cell response, or identify properties predictive for the efficacy of antibodies in vitro has been reported.

The key technology for high-throughput data acquisition for this purpose is imaging flow cytometry (IFC), combining the benefits of traditional flow cytometry with deep, multi-channel imaging on the single-cell level. IFC has recently been successfully applied to visualize and quantify the immunological synapse of primary human T: APC cell conjugates [19-21], however, none of these studies investigated the formation of the immunological synapse in the context of T-cell function.

Recent studies have demonstrated the potential of machine learning algorithms for a more robust and accurate analysis of high-throughput imaging data, an approach that has been demonstrated to overcome limitations of conventional gating strategies [22-24]. Leveraging machine learning for IFC data analysis has also enabled the identification of morphological patterns in the cell, a combined analysis of RNA and protein data, and the implementation of predictive models [22-26]. While limited open-source software implementations designed for IFC data analysis are available [26,27], they either rely on additional software adding complexity in the analysis pipeline, or they focus on prediction performance only and lack explainability.

The immunological synapse has previously been studied using high-content cell imaging on human cell lines and primary cells with an artificial APC system that utilized plate-bound ICAM-1 and stimulatory antibodies [37]. Although German et al. convincingly demonstrated the capabilities of their pipeline by profiling the immunological synapse, they did not investigate whether they can use these profiles in predicting drug effectiveness [37]. In other studies, the potential of synapse formation was also investigated for CAR T-cell therapy, where investigators used the mean intensity of stainings such as F-actin and P-CD3zeta per cell, clustering of tumor antigen and polarization of perforin-containing granules as a measure of synapse formation quality. These features varied between different CAR T-cells and correlated with their effectiveness in vitro and in vivo as well as with clinical outcomes [39,40].

M. Chen et al. disclose that heparin-binding EGF-like growth factor modulates the bidirectional activation of CD4+ T-cells and dendritic cells independently of the Epidermal Growth Factor Receptor (Am. J. Resp. Crit. Care, 2018, MeetingAbstracts.A5826).

B. H. Hosseini et al. disclose that immune synapse formation determines interaction forces between T-cells and antigen-presenting cells measured by atomic force microscopy (Proc. Natl. Acad. Sci USA 106 (2009) 17852-17857).

F. Ahmed et al. disclose that numbers matter in quantitative and dynamic analysis of the formation of an immunological synapse using imaging flow cytometry (J. Immunol. Meth. 347 (2009) 79-86).

G. Wabnitz et al. disclose that inflow microscopy of human leukocytes is a tool for quantitative analysis of actin rearrangements in the immune synapse (J. Immunol. Meth. 423 (2015) 29-39).

US 2021/270812 discloses method for analyzing immune cells.

### SUMMARY OF THE INVENTION

Herein is reported a method for classifying cells in a cell mixture using single-cell imaging flow cytometry.

Herein is further reported a method for classifying cells in a cell mixture using single-cell imaging flow cytometry in combination with artificial intelligence (scifAI) for preprocessing, feature engineering and explainable, predictive machine learning on imaging flow cytometry (IFC) data. An algorithmic flowchart is presented in FIG. 40.

With the methods according to the invention it is possible to analyze class frequency as well as morphological changes under different immune stimulation. The applicability of the methods according to the invention have been shown by analyzing T-cell cytokine production across multiple donors and therapeutic antibodies. The characteristics have been quantitatively predicted in vitro, linking morphological features with function and demonstrating the potential to significantly impact antibody design.

The methods according to the invention are universally applicable to IFC data, and, given its modular architecture, straightforward to incorporate into existing workflows and analysis pipelines, e.g. for rapid antibody screening and functional characterization.

Thus, the current invention encompasses the following embodiments:
1. A method for classifying cells in a cell mixture, wherein the mixture comprises T-cells and activated B-cells or antigen-presenting cells, comprising the following steps:
   a) applying at least labelled antibodies binding to F-actin, MHCII and CD3 to the cell mixture to obtained a labelled cell mixture, wherein the antibodies are each labelled with a dye, wherein the dyes have different, optionally non-overlapping, emission wavelengths,
   b) acquiring at least one image of the cell mixture, and
   c) classifying the cells in the cell mixture to be
      i) an isolated cell if the cell is a single cell, is F-Actin positive and
         is MHCII positive and CD3 negative, or
         is MHCII negative and CD3 positive,
      ii) a doublet or multiplet of cells if the cell is an aggregate of two or three or more cells, is F-Actin positive, MHCII positive and CD3 positive.
1a. A method for the classification of cells in a cell mixture, wherein the mixture comprises T-cells and activated B-cells or antigen-presenting cells, comprising the following steps:
   a) applying at least labelled antibodies binding to F-actin, MHCII and CD3 to the cell mixture to obtained a labelled cell mixture, wherein the antibodies are each labelled with a dye, wherein the dyes have different, optionally non-overlapping, emission wavelengths,
   b) acquiring at least one image of the cell mixture, and
   c) classifying the image as follows:
      the image contains
      i) an isolated cell if the cell in the image is F-Actin positive and
         is MHCII positive and CD3 negative, or
         is MHCII negative and CD3 positive,
      ii) a doublet or multiplet of cells if the cell in the image are an aggregate of two or three or more cells and the aggregate is F-Actin positive, MHCII positive and CD3 positive.
2. The method according to embodiment 1, wherein
   step a) is: applying at least labelled antibodies binding to F-actin, MHCII, CD3 and P-CD3zeta to the cell mixture, wherein the antibodies are each labelled with a dye, wherein the dyes have different (non-overlapping) emission wavelengths,
   and step c) is: classifying the cells in the cell mixture to be
      i) a single B-cell or antigen-presenting cell if the cell is F-Actin positive, MHCII positive, CD3 negative and P-CD3zeta negative,
      ii) a single T-cell without signaling if the cell is F-Actin positive, MHCII negative, CD3 positive and P-CD3zeta negative,
      iii) a single T-cell with signaling if the cell is F-Actin positive, MHCII negative, CD3 positive and P-CD3zeta positive,
      iv) a doublet of a B-cell or antigen-presenting cell and a T-cell forming a synapse without signaling if the doublet is F-Actin positive, MHCII positive, CD3 positive and P-CD3zeta negative,
      v) a doublet or multiplet of one or two or more B-cells or antigen-presenting cells and one or two or more T-cells forming a synapse with signaling if the doublet or multiplet is F-Actin positive, MHCII positive, CD3 positive and P-CD3zeta positive.
2a. The method according to embodiment 1a, wherein
   step a) is: applying at least labelled antibodies binding to F-actin, MHCII, CD3 and P-CD3zeta to the cell mixture, wherein the antibodies are each labelled with a dye, wherein the dyes have different (non-overlapping) emission wavelengths,
   and step c) is: classifying the image as follows:
      the image contains
         i) a single B-cell or antigen-presenting cell if the cell in the image is F-Actin positive, MHCII positive, CD3 negative and P-CD3zeta negative,
         ii) a single T-cell without signaling if the cell in the image is F-Actin positive, MHCII negative, CD3 positive and P-CD3zeta negative,
         iii) a single T-cell with signaling if the cell in the image is F-Actin positive, MHCII negative, CD3 positive and P-CD3zeta positive,
         iv) a doublet of a B-cell or antigen-presenting cell and a T-cell forming a synapse without signaling if the cell doublet in the image is F-Actin positive, MHCII positive, CD3 positive and P-CD3zeta negative,
         v) a doublet or multiplet of one or two or more B-cells or antigen-presenting cells and one or two or more T-cells forming a synapse with signaling if the cell doublet or cell multiplet in the image is F-Actin positive, MHCII positive, CD3 positive and P-CD3zeta positive.
3. The method according to any one of embodiments 1 to 2a, wherein step b) is: acquiring images of the cell mixture using a imaging flow cytometer.
4. The method according to any one of embodiments 1 to 3, wherein the acquired images are images each showing a single cell or isolated doublets or multiplets.
5. A method for classifying cells in a cell mixture, wherein the mixture comprises T-cells and activated B-cells or antigen-presenting cells, comprising the following steps:
   a) acquiring at least one image of the cell mixture,
   b) generating a feature extraction pipeline to derive biologically interpretable features from the at least one image,
   c) predicting based on the derived biologically interpretable features the cell to be in one of the classes
      i) a single B-cell or antigen-presenting cell,
      ii) a single T-cell without signaling,
      iii) a single T-cell with signaling,
      iv) a doublet of a B-cell or antigen-presenting cell and a T-cell forming a synapse without signaling,
      v) a doublet or multiplet of one or two or more B-cells or antigen-presenting cells and one or two or more T-cells forming a synapse with signaling.
5a. A method for classifying cells in a cell mixture, wherein the mixture comprises T-cells and activated B-cells or antigen-presenting cells, comprising the following steps:
   a) acquiring at least one image of the cell mixture,
   b) generating a feature extraction pipeline to derive biologically interpretable features from the at least one image,
   c) predicting based on the derived biologically interpretable features the cell in the image to be
      i) a single B-cell or antigen-presenting cell,
      ii) a single T-cell without signaling,
      iii) a single T-cell with signaling,
      iv) a doublet of a B-cell or antigen-presenting cell and a T-cell forming a synapse without signaling,
      v) a doublet or multiplet of one or two or more B-cells or antigen-presenting cells and one or two or more T-cells forming a synapse with signaling.
6. The method according to any one of embodiments 1 to 5a, wherein the doublets and multiplets of cells (in the image(s)) are classified to be a synapse based on morphology, labelling intensity, co-localization of labels, texture and synaptic features.
7. The method according to any one of embodiments 1 to 6, wherein the doublets and multiplets of cells (in the image(s)) are classified to be a synapse based on one or more of the following further features
   the colocalization of the CD3 and MHCII labelling, or/and
   the colocalization of the MHCII and P-CD3zeta labelling, or/and
   the texture of the MHCII, or/and
   the texture of the CD3 labelling, or/and
   the intensity of the P-CD3zeta labelling.
8. The method according to any one of embodiments 1 to 7, wherein the features are determined for each cell or doublet or multiplet or image based on the ratio of the label signal intensity in the synaptic area to the whole cell.
9. The method according to any one of embodiments 1 to 4 and 6 to 8, wherein the labelled cell mixture is an intracellularly labelled cell mixture obtained by fixing the cells, permealizing the cells and applying the labelled antibodies.
9a. The method according to any one of embodiments 1 to 9, wherein the method further comprises the following step d)
   d) counting the number of cells or images of cells in each class and calculating a relative frequency of the cells of each class in the cell mixture.
10. The method according to any one of embodiments 1 to 9a, wherein dead, deformed or cropped cells (aggregates of more than three cells) are removed prior to step b), or wherein no image of dead, deformed or cropped cells (aggregates of more than three cells) is recorded, or wherein images of dead, deformed or cropped cells, as well as unfocussed images are removed prior to step c), or wherein images of dead, deformed or cropped cells, as well as unfocussed images are not analyzed in step c), or wherein images of dead, deformed or cropped cells, as well as unfocussed images are not counted in step d).
11. The method according to any one of embodiments 1 to 10, wherein step b) comprises the following additional sub-steps:
   b-1) gating of in-focus live+ CD3+ MHCII+ cells,
   b-2) selecting from the population obtained in step b-1) images that show single CD3+ T-cells and single MHCII+ B-cells or antigen presenting cells using the area and aspect ratio feature,
   b-3) determining the signal intensity of the labelled CD3 within the synapse mask (the synapse mask is defined as a combination of the morphology CD3 and MHCII mask with a dilation of 3) and gating synapses showing a CD3 signal in the mask, and
   b-4) excluding (the image of) T-cells and B-cells or antigen-presenting cells in one layer by using the height and area feature of the brightfield (BF).
12. The method according to any one of embodiments 5 to 11, wherein the method comprises as step c):
   c) building a model based on the derived biologically interpretable features based on an XGBoost classifier and predicting based on the model the cell or the image to be in one of the classes of
      i) a single B-cell or antigen-presenting cell,
      ii) a single T-cell without signaling,
      iii) a single T-cell with signaling,
      iv) a doublet of a B-cell or antigen-presenting cell and a T-cell forming a synapse without signaling,
      v) a doublet or multiplet of one or two or more B-cells or antigen-presenting cells and one or two or more T-cells forming a synapse with signaling.
13. The method according to any one of embodiments 1 to 12, wherein the mixture comprises B-cells or antigen-presenting cells and T-cells at a cellular ratio of about 4:3.
14. The method according to any one of embodiments 1 to 13, wherein the T-cells are CD4 positive memory T-cells or CD8 positive T-cells or mixtures thereof.
15. The method according to any one of embodiments 1 to 14, wherein the T-cells are CD4 positive memory T-cells.
16. The method according to any one of embodiments 1 to 15, wherein the mixture of cells is centrifuged after the mixing.
17. The method according to any one of embodiments 1 to 16, wherein step b) further comprises compensating the images using a compensation matrix derived from stained single cells.
18. A method for classifying cells in a cell mixture, wherein the mixture comprises T-cells and activated B-cells or antigen-presenting cells, comprising the following steps
   a) preparation of a labelled cell mixture by
      a-1) aliquoting the cell mixture in at least two aliquots,
      a-2) applying to a first aliquot of the mixture an antibody that binds to one or more cell surface targets present on one or both of the cells of the mixture, and applying to a second aliquot of the mixture an antibody that has the same structure as the antibody applied to the first aliquot but does not bind to one or more cell surface targets present on one or both of the cells of the mixture,
      a-3) incubating the aliquots obtained in step a-2),
      a-4) applying at least labelled antibodies binding to F-actin, MHCII and CD3 to the incubated aliquots of the cell mixture obtained in step a-3) to obtained a labelled cell mixture, wherein the antibodies are each labelled with a dye, wherein the dyes have different (non-overlapping) emission wavelengths,
   b) acquiring at least one image of each aliquot of the labelled cell mixture, and
   c) separately classifying the images of the cells in each aliquot of the cell mixture to comprise
      i) an isolated cell if the image comprises a cell that is a single cell, is F-Actin positive and
         is MHCII positive and CD3 negative, or
         is MHCII negative and CD3 positive,
      ii) a doublet or multiplet of cells if the image comprises cells that are an aggregate of two or three cells, are F-Actin positive, MHCII positive and CD3 positive.
   d) counting for each aliquot the number of images of cells in each class and calculating a relative frequency of the cells of each class in the cell mixture, and
   e) determining the difference in the class frequencies between the first and the second aliquot.
19. The method according to embodiment 18, wherein
   step a-4) is: applying at least labelled antibodies binding to F-actin, MHCII, CD3 and P-CD3zeta to the incubated aliquots of the cell mixture, wherein the antibodies are each labelled with a dye, wherein the dyes have different (non-overlapping) emission wavelengths,
   and step c) is: classifying the cells in the cell mixture to be
      i) a single B-cell or antigen-presenting cell if the cell is F-Actin positive, MHCII positive, CD3 negative and P-CD3zeta negative,
      ii) a single T-cell without signaling if the cell is F-Actin positive, MHCII negative, CD3 positive and P-CD3zeta negative,
      iii) a single T-cell with signaling if the cell is F-Actin positive, MHCII negative, CD3 positive and P-CD3zeta positive,
      iv) a doublet of a B-cell or antigen-presenting cell and a T-cell forming a synapse without signaling if the doublet is F-Actin positive, MHCII positive, CD3 positive and P-CD3zeta negative,
      v) a doublet or multiplet of one or two or more B-cells or antigen-presenting cells and one or two or more T-cells forming a synapse with signaling if the doublet or multiplet is F-Actin positive, MHCII positive, CD3 positive and P-CD3zeta positive.
19a. The method according to embodiment 18, wherein
   step a-4) is: applying at least labelled antibodies binding to F-actin, MHCII, CD3 and P-CD3zeta to the incubated aliquots of the cell mixture, wherein the antibodies are each labelled with a dye, wherein the dyes have different (non-overlapping) emission wavelengths,
   and step c) is: classifying the image as follows:
      the image contains
      i) a single B-cell or antigen-presenting cell if the cell in the image is F-Actin positive, MHCII positive, CD3 negative and P-CD3zeta negative,
      ii) a single T-cell without signaling if the cell in the image is F-Actin positive, MHCII negative, CD3 positive and P-CD3zeta negative,
      iii) a single T-cell with signaling if the cell in the image is F-Actin positive, MHCII negative, CD3 positive and P-CD3zeta positive,
      iv) a doublet of a B-cell or antigen-presenting cell and a T-cell forming a synapse without signaling if the doublet in the image is F-Actin positive, MHCII positive, CD3 positive and P-CD3zeta negative,
      v) a doublet or multiplet of one or two or more B-cells or antigen-presenting cells and one or two or more T-cells forming a synapse with signaling if the doublet or multiplet in the image is F-Actin positive, MHCII positive, CD3 positive and P-CD3zeta positive.
20. The method according to any one of embodiments 18 to 19a, wherein
   step b) is: acquiring images of the cell mixture using a imaging flow cytometer.
21. The method according to any one of embodiments 18 to 20, wherein the acquired images are images each showing a single cell or isolated doublets or multiplets.
22. The method according to any one of embodiments 18 to 21, wherein the doublets and multiplets of cells or images thereof are classified to be a synapse based on morphology, labelling intensity, co-localization of labels, texture and synaptic features.
23. The method according to any one of embodiments 18 to 22, wherein the doublets and multiplets of cells in the image are classified to be a synapse based on one or more of the following further features
   the colocalization of the CD3 and MHCII labelling, or/and
   the colocalization of the MHCII and P-CD3zeta labelling, or/and
   the texture of the MHCII, or/and
   the texture of the CD3 labelling, or/and
   the intensity of the P-CD3zeta labelling.
24. The method according to any one of embodiments 18 to 23, wherein the features are determined for each cell or doublet or multiplet or image based on the ratio of the label signal intensity in the synaptic area to the whole cell.
25. The method according to any one of embodiments 18 to 24, wherein the labelled cell mixture is an intracellularly labelled cell mixture obtained by fixing the cells, permealizing the cells and applying the labelled antibodies.
26. The method according to any one of embodiments 18 to 25, wherein dead, deformed or cropped cells (aggregates of more than three cells) are removed prior to step b), or wherein no image of dead, deformed or cropped cells (aggregates of more than three cells) is recorded, or wherein images of dead, deformed or cropped cells, as well as unfocussed images are removed prior to step c), or wherein images of dead, deformed or cropped cells, as well as unfocussed images are not analyzed in step c), or wherein images of dead, deformed or cropped cells, as well as unfocussed images are not counted in step d).
27. The method according to any one of embodiments 18 to 26, wherein step b) comprises the following additional sub-steps:
   b-1) gating of in-focus live+ CD3+ MHCII+ cells,
   b-2) selecting from the population obtained in step b-1) images that show single CD3+ T-cells and single MHCII+ B-cells or antigen presenting cells using the area and aspect ratio feature,
   b-3) determining the signal intensity of the labelled CD3 within the synapse mask (the synapse mask is defined as a combination of the morphology CD3 and MHCII mask with a dilation of 3) and gating synapses showing a CD3 signal in the mask, and
   b-4) excluding T-cells and B-cells or antigen-presenting cells in one layer by using the height and area feature of the brightfield (BF).
28. The method according to any one of embodiments 18 to 27, wherein the mixture comprises B-cells or antigen-presenting cells and T-cells at a cellular ratio of about 4:3.
29. The method according to any one of embodiments 18 to 28, wherein the T-cells are CD4 positive memory T-cells or CD8 positive T-cells or mixtures thereof.
30. The method according to any one of embodiments 18 to 29, wherein the T-cells are CD4 positive memory T-cells.
31. The method according to any one of embodiments 18 to 30, wherein the mixture of cells is centrifuged after the mixing.
32. The method according to any one of embodiments 18 to 31, wherein step b) further comprises compensating the images using a compensation matrix derived from stained single cells.
33. A method for ranking antibodies in a multitude of antibodies, comprising the following steps:
   1) performing the method according to any one of embodiments 18 to 31 individually for each antibody of the multitude of antibodies or together for all antibodies with each antibody being applied to a separate aliquot of the mixture,
   2) ranking the antibodies based on the change of frequency of one or more of the classes (of images) of
      i) single B-cell or antigen-presenting cell,
      ii) single T-cell without signaling,
      iii) single T-cell with signaling,
      iv) doublet of a B-cell or antigen-presenting cell and a T-cell forming a synapse without signaling, and
      v) doublet or multiplet of one or two or more B-cells or antigen-presenting cells and one or two or more T-cells forming a synapse with signaling.
34. The method according to embodiment 33, wherein the ranking of the antibodies in the multitude of antibodies is with decreasing stimulation of immune response.
35. The method according to any one of embodiments 33 to 34, wherein the ranking of the antibodies in the multitude of antibodies is by decreasing frequency of the cells or images classified as doublet or multiplet of one or two or more B-cells or antigen-presenting cells and one or two or more T-cells forming a synapse with signaling.
36. The method according to any one of embodiments 33 to 35, wherein the ranking of the antibodies in the multitude of antibodies is further by decreasing signal intensity of the labelled F-Actin, the labelled P-CD3zeta and the labelled MHCII in the synaptic area.
37. The method according to embodiment 33, wherein the ranking of the antibodies in the multitude of antibodies is with decreasing inhibition of immune response.
38. The method according to any one of embodiments 33 and 37, wherein the ranking of the antibodies in the multitude of antibodies is by decreasing frequency of the cells or images classified as doublet or multiplet of one or two or more B-cells or antigen-presenting cells and one or two or more T-cells forming a synapse with signaling.
39. The method according to any one of embodiments 33 and 37 to 38, wherein the ranking of the antibodies in the multitude of antibodies is further by increasing frequency of the cells or images classified as single T-cell without signaling.
40. The method according to any one of embodiments 33 and 37 to 39, wherein the ranking of the antibodies in the multitude of antibodies is further by decreasing frequency of the cells or images classified as single T-cell with signaling.
41. The method according to any one of embodiments 33 and 37 to 40, wherein the ranking of the antibodies in the multitude of antibodies is further by increasing frequency of the mean signal intensity of the labelled F-Actin, by increasing signal of the intensity of the labelled P-CD3zeta within the synaptic area, and by de-clustering of the signal for the T-cell receptor.
42. Use of the method according to any one of embodiments 1 to 41 for characterizing the morphology of a synapse formed between a T-cell and a B-cell or antigen-presenting cell.
43. Use of the method according to any one of embodiments 33 to 41 for determining the correlation between antibody concentration and T-cell response.
44. Use of the method according to any one of embodiments 33 to 41 for predicting therapeutic mode of action of an antibody.
45. Use of the method according to any one of embodiments 33 to 41 for predicting efficacy of an antibody.
46. Use of F-actin, MHCII, and CD3 for classifying T-cells in a mixture comprising T-cells and B-cells or activated B-cells or antigen-presenting cells.
47. Use of F-actin, MHCII, and CD3 for classifying B-cells in a mixture comprising T-cells and B-cells or activated B-cells or antigen-presenting cells.
48. The use according to any one of embodiments 46 to 47, wherein the image contains or the cell is classified to be an isolated cell, i.e. the classifying of the cell is that the cell is an isolated cell, if the cell is a single cell, is F-Actin positive and
   is MHCII positive and CD3 negative, or
   is MHCII negative and CD3 positive.
49. The use according to any one of embodiments 46 to 47, wherein the image contains or the cell is classified to be a doublet or multiplet of cells, i.e. the classifying of the cell is that the cell is a doublet or multiplet of cells, if the cell is an aggregate of two or three cells, is F-Actin positive, MHCII positive and CD3 positive.
50. The use according to any one of embodiments 46 to 49, further comprising P-CD3zeta.
51. The use according to any one of embodiments 46 to 50, wherein the image contains or the cell is classified to be a single B-cell or antigen-presenting cell, i.e. the classifying of the cell is that the cell is a single B-cell or antigen-presenting cell, if the cell is F-Actin positive, MHCII positive, CD3 negative and P-CD3zeta negative.
52. The use according to any one of embodiments 46 to 51, wherein the image contains or the cell is classified to be a single T-cell without signaling, i.e. the classifying of the cell is that the cell is a single T-cell without signaling, if the cell is F-Actin positive, MHCII negative, CD3 positive and P-CD3zeta negative.
53. The use according to any one of embodiments 46 to 52, wherein the image contains or the cell is classified to be a single T-cell with signaling, i.e. the classifying of the cell is that the cell is a single T-cell with signaling, if the cell is F-Actin positive, MHCII negative, CD3 positive and P-CD3zeta positive.
54. The use according to any one of embodiments 46 to 53, wherein the image contains or the cell is classified to be a doublet of a B-cell or antigen-presenting cell and a T-cell forming a synapse without signaling, i.e. the classifying of the cell is that the cell is a doublet of a B-cell or antigen-presenting cell and a T-cell forming a synapse without signaling, if the doublet is F-Actin positive, MHCII positive, CD3 positive and P-CD3zeta negative.
55. The use according to any one of embodiments 46 to 54, wherein the image contains or the cell is classified is to be a doublet or multiplet of one or two or more B-cells or antigen-presenting cells and one or two or more T-cells forming a synapse with signaling, i.e. the classifying of the cell is that the cell is a doublet or multiplet of one or two or more B-cells or antigen-presenting cells and one or two or more T-cells forming a synapse with signaling, if the doublet or multiplet is F-Actin positive, MHCII positive, CD3 positive and P-CD3zeta positive.
56. The method or use according to any one of embodiments 1 to 55, wherein the multiplet is a multiplet of two B-cells or antigen-presenting cells and one T-cell
57. The method or use according to any one of embodiments 1 to 55, wherein the multiplet is a multiplet of one B-cell or antigen-presenting cell and two T-cells.

In addition to the various embodiments depicted and claimed, the disclosed subject matter is also directed to other embodiments having other combinations of the features disclosed and claimed herein. As such, the particular features presented herein can be combined with each other in other manners within the scope of the disclosed subject matter such that the disclosed subject matter includes any suitable combination of the features disclosed herein. The foregoing description of specific embodiments of the disclosed subject matter has been presented for purposes of illustration and description. It is not intended to be exhaustive or to limit the disclosed subject matter to those embodiments disclosed.

### DETAILED DESCRIPTION OF THE INVENTION

### General Definitions

It must be noted that as used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably.

The term “about” denotes a range of +/−20% of the thereafter following numerical value. In certain embodiments, the term about denotes a range of +/−10% of the thereafter following numerical value. In certain embodiments, the term about denotes a range of +/−5% of the thereafter following numerical value.

The terms “comprise(s),” “include(s),” “having,” “has,” “can,” “contain(s)” and variants thereof, as used herein, are intended to be open-ended transitional phrases, terms or words that do not preclude the possibility of additional acts or structures. The term “comprising” also encompasses the term “consisting of”. The present disclosure also contemplates other embodiments “comprising,” “consisting of” and “consisting essentially of,” the embodiments or elements presented herein, whether explicitly set forth or not.

### Antibodies

General information regarding the nucleotide sequences of human immunoglobulins light and heavy chains is given in: Kabat, E. A., et al., Sequences of Proteins of Immunological Interest, 5th ed., Public Health Service, National Institutes of Health, Bethesda, MD (1991).

The term “antibody” herein is used in the broadest sense and encompasses various antibody structures, including but not limited to full-length antibodies, monoclonal antibodies, multispecific antibodies (e.g., bispecific antibodies), and antibody-antibody fragment-fusions as well as combinations thereof.

The term “native antibody” denotes naturally occurring immunoglobulin molecules with varying structures. For example, native IgG antibodies are heterotetrameric glycoproteins of about 150,000 Daltons, composed of two identical light chains and two identical heavy chains that are disulfide-bonded. From N- to C-terminus, each heavy chain has a heavy chain variable region (VH) followed by three heavy chain constant domains (CH1, CH2, and CH3), whereby between the first and the second heavy chain constant domain a hinge region is located. Similarly, from N- to C-terminus, each light chain has a light chain variable region (VL) followed by a light chain constant domain (CL). The light chain of an antibody may be assigned to one of two types, called kappa (κ) and lambda (λ), based on the amino acid sequence of its constant domain.

The term “full-length antibody” denotes an antibody having a structure substantially similar to that of a native antibody. A full length antibody comprises two full length antibody light chains each comprising in N- to C-terminal direction a light chain variable region and a light chain constant domain, as well as two full length antibody heavy chains each comprising in N- to C-terminal direction a heavy chain variable region, a first heavy chain constant domain, a hinge region, a second heavy chain constant domain and a third heavy chain constant domain. In contrast to a native antibody, a full length antibody may comprise further immunoglobulin domains, such as e.g. one or more additional scFvs, or heavy or light chain Fab fragments, or scFabs conjugated to one or more of the termini of the different chains of the full length antibody, but only a single fragment to each terminus. These conjugates are also encompassed by the term full-length antibody.

The “class” of an antibody refers to the type of constant domains or constant region, preferably the Fc-region, possessed by its heavy chains. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called a, 8, ¿, Y, and u, respectively.

The term “heavy chain constant region” denotes the region of an immunoglobulin heavy chain that contains the constant domains, i.e. the CH1 domain, the hinge region, the CH2 domain and the CH3 domain. In certain embodiments, a human IgG constant region extends from Alal 18 to the carboxyl-terminus of the heavy chain (numbering according to Kabat EU index). However, the C-terminal lysine (Lys447) of the constant region may or may not be present (numbering according to Kabat EU index). The term “constant region” denotes a dimer comprising two heavy chain constant regions, which can be covalently linked to each other via the hinge region cysteine residues forming inter-chain disulfide bonds.

The term “heavy chain Fc-region” denotes the C-terminal region of an immunoglobulin heavy chain that contains at least a part of the hinge region (middle and lower hinge region), the CH2 domain and the CH3 domain. In certain embodiments, a human IgG heavy chain Fc-region extends from Asp221, or from Cys226, or from Pro230, to the carboxyl-terminus of the heavy chain (numbering according to Kabat EU index). Thus, an Fc-region is smaller than a constant region but in the C-terminal part identical thereto. However, the C-terminal lysine (Lys447) of the heavy chain Fc-region may or may not be present (numbering according to Kabat EU index). The term “Fc-region” denotes a dimer comprising two heavy chain Fc-regions, which can be covalently linked to each other via the hinge region cysteine residues forming inter-chain disulfide bonds.

The constant region, more precisely the Fc-region, of an antibody (and the constant region likewise) is directly involved in complement activation, C1q binding, C3 activation and Fc receptor binding. While the influence of an antibody on the complement system is dependent on certain conditions, binding to C1q is caused by defined binding sites in the Fc-region. Such binding sites are known in the state of the art and described e.g. by Lukas, T. J., et al., J. Immunol. 127 (1981) 2555-2560; Brunhouse, R., and Cebra, J. J., Mol. Immunol. 16 (1979) 907-917: Burton, D. R., et al., Nature 288 (1980) 338-344; Thommesen, J. E., et al., Mol. Immunol. 37 (2000) 995-1004: Idusogie, E. E., et al., J. Immunol. 164 (2000) 4178-4184; Hezareh, M., et al., J. Virol. 75 (2001) 12161-12168: Morgan, A., et al., Immunology 86 (1995) 319-324; and EP 0 307 434. Such binding sites are e.g. L234, L235, D270, N297, E318, K320, K322, P331 and P329 (numbering according to EU index of Kabat).

Antibodies of subclass IgG1, IgG2 and IgG3 usually show complement activation, C1q binding and C3 activation, whereas IgG4 do not activate the complement system, do not bind C1q and do not activate C3. An “Fc-region of an antibody” is a term well known to the skilled artisan and defined on the basis of papain cleavage of antibodies.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, monoclonal antibodies may be made by a variety of techniques, including but not limited to the hybridoma method, recombinant DNA methods, phage-display methods, and methods utilizing transgenic animals containing all or part of the human immunoglobulin loci.

The term “valent” as used within the current application denotes the presence of a specified number of binding sites in an antibody. As such, the terms “bivalent”, “tetravalent”, and “hexavalent” denote the presence of two binding site, four binding sites, and six binding sites, respectively, in an antibody.

A “monospecific antibody” denotes an antibody that has a single binding specificity, i.e. specifically binds to one antigen. Monospecific antibodies can be prepared as full-length antibodies or antibody fragments (e.g. F(ab′)2) or combinations thereof (e.g. full length antibody plus additional scFv or Fab fragments). A monospecific antibody does not need to be monovalent, i.e. a monospecific antibody may comprise more than one binding site specifically binding to the one antigen. A native antibody, for example, is monospecific but bivalent.

A “multispecific antibody” denotes an antibody that has binding specificities for at least two different epitopes on the same antigen or two different antigens. Multispecific antibodies can be prepared as full-length antibodies or antibody fragments (e.g. F(ab′)2 bispecific antibodies) or combinations thereof (e.g. full length antibody plus additional scFv or Fab fragments). A multispecific antibody is at least bivalent, i.e. comprises two antigen binding sites. In addition, a multispecific antibody is at least bispecific. Thus, a bivalent, bispecific antibody is the simplest form of a multispecific antibody. Engineered antibodies with two, three or more (e.g. four) functional antigen binding sites have also been reported (see, e.g., US 2002/0004587).

In certain embodiments of all aspects and embodiments of the invention, the antibody is a multispecific antibody, e.g. at least a bispecific antibody. In certain embodiments, one of the binding specificities is for a first antigen and the other is for a different second antigen. In certain embodiments, multispecific antibodies may bind to two different epitopes of the same antigen. Multispecific antibodies may also be used to localize cytotoxic agents to cells, which express the one or more antigens.

Multispecific antibodies can be prepared as full-length antibodies or antibody-antibody fragment-fusions.

Techniques for making multispecific antibodies include, but are not limited to, recombinant co-expression of two immunoglobulin heavy chain-light chain pairs having different specificities (see Milstein, C. and Cuello, A. C., Nature 305 (1983) 537-540, WO 93/08829, and Traunecker, A., et al., EMBO J. 10 (1991) 3655-3659), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004): cross-linking two or more antibodies or fragments (see, e.g., U.S. Pat. No. 4,676,980, and Brennan, M., et al., Science 229 (1985) 81-83): using leucine zippers to produce bi-specific antibodies (see, e.g., Kostelny, S. A., et al., J. Immunol. 148 (1992) 1547-1553): using the common light chain technology for circumventing the light chain mis-pairing problem (see, e.g., WO 98/50431): using specific technology for making bispecific antibody fragments (see, e.g., Holliger, P., et al., Proc. Natl. Acad. Sci. USA 90 (1993) 6444-6448); and preparing trispecific antibodies as described, e.g., in Tutt, A., et al., J. Immunol. 147 (1991) 60-69).

Engineered antibodies with three or more antigen binding sites, including for example, “Octopus antibodies”, or DVD-Ig are also included herein (see, e.g., WO 2001/77342 and WO 2008/024715). Other examples of multispecific antibodies with three or more antigen binding sites can be found in WO 2010/115589, WO 2010/112193, WO 2010/136172, WO 2010/145792, and WO 2013/026831. The bispecific antibody or antigen binding fragment thereof also includes a “Dual Acting Fab” or “DAF” (see, e.g., US 2008/0069820 and WO 2015/095539).

Multi-specific antibodies may also be provided in an asymmetric form with a domain crossover, i.e. by exchanging the VH/VL domains (see, e.g., WO 2009/080252 and WO 2015/150447), the CH1/CL domains (see, e.g., WO 2009/080253) or the complete Fab arms (see e.g., WO 2009/080251, WO 2016/016299, also see Schaefer et al., Proc. Natl. Acad. Sci. USA 108 (2011) 1187-1191, and Klein at al., MAbs 8 (2016) 1010-1020) in one or more binding arms of the same antigen specificity. In certain embodiments of all aspects and embodiments of the invention, the multispecific antibody comprises a Cross-Fab fragment. The term “Cross-Fab fragment” denotes a Fab fragment, wherein either the variable regions or the constant regions of the heavy and light chain are exchanged. A Cross-Fab fragment comprises a polypeptide chain composed of the light chain variable region (VL) and the heavy chain constant region 1 (CH1), and a polypeptide chain composed of the heavy chain variable region (VH) and the light chain constant region (CL). Asymmetrical Fab arms can also be engineered by introducing charged or non-charged amino acid mutations into domain interfaces to direct correct Fab heavy chain fragment and cognate light chain pairing. See, e.g., WO 2016/172485.

The antibody or fragment may also be a multispecific antibody as described in WO 2009/080254, WO 2010/112193, WO 2010/115589, WO 2010/136172, WO 2010/145792, or WO 2010/145793.

The antibody or fragment thereof may also be a multispecific antibody as disclosed in WO 2012/163520.

Various further molecular formats for multispecific antibodies are known in the art and can be produced using a cell according to the current invention (see e.g., Spiess et al., Mol. Immunol. 67 (2015) 95-106).

Bispecific antibodies are generally antibody molecules that specifically bind to two different, non-overlapping epitopes on the same antigen or to two epitopes on different antigens.

In certain embodiments of all aspects and embodiments, the antibody is a complex (multispecific) antibodies selected from the group of complex (multispecific) antibodies consisting of
a full-length antibody with domain exchange
   (i.e. a multispecific IgG antibody comprising a first Fab fragment and a second Fab fragment, wherein in the first Fab fragment
   a) only the CH1 and CL domains are replaced by each other (i.e. the light chain of the first Fab fragment comprises a VL and a CH1 domain and the heavy chain of the first Fab fragment comprises a VH and a CL domain); b) only the VH and VL domains are replaced by each other (i.e. the light chain of the first Fab fragment comprises a VH and a CL domain and the heavy chain of the first Fab fragment comprises a VL and a CH1 domain); or
   c) the CH1 and CL domains are replaced by each other and the VH and VL domains are replaced by each other (i.e. the light chain of the first Fab fragment comprises a VH and a CH1 domain and the heavy chain of the first Fab fragment comprises a VL and a CL domain); and
   wherein the second Fab fragment comprises a light chain comprising a VL and a CL domain, and a heavy chain comprising a VH and a CH1 domain:
   the full-length antibody with domain exchange may comprises a first heavy chain including a CH3 domain and a second heavy chain including a CH3 domain, wherein both CH3 domains are engineered in a complementary manner by respective amino acid substitutions, in order to support heterodimerization of the first heavy chain and the modified second heavy chain, e.g. as disclosed in WO 96/27011, WO 98/050431, EP 1870459, WO 2007/110205, WO 2007/147901, WO 2009/089004, WO 2010/129304, WO 2011/90754, WO 2011/143545, WO 2012/058768, WO 2013/157954, or WO 2013/096291 (incorporated herein by reference)):
a full-length antibody with domain exchange and additional heavy chain C-terminal binding site (BS)
   (i.e. a multispecific IgG antibody comprising
   a) one full length antibody comprising two pairs each of a full length antibody light chain and a full length antibody heavy chain, wherein the binding sites formed by each of the pairs of the full length heavy chain and the full length light chain specifically bind to a first antigen, and
   b) one additional Fab fragment, wherein the additional Fab fragment is fused to the C-terminus of one heavy chain of the full length antibody, wherein the binding site of the additional Fab fragment specifically binds to a second antigen,
   wherein the additional Fab fragment specifically binding to the second antigen i) comprises a domain crossover such that a) the light chain variable domain (VL) and the heavy chain variable domain (VH) are replaced by each other, or b) the light chain constant domain (CL) and the heavy chain constant domain (CH1) are replaced by each other, or ii) is a single chain Fab fragment):
a one-armed single chain antibody
   (i.e. an antibody comprising a first binding site that specifically binds to a first epitope or antigen and a second binding site that specifically binds to a second epitope or antigen, whereby the individual chains are as follows
      light chain (variable light chain domain+light chain kappa constant domain)
      combined light/heavy chain (variable light chain domain+light chain constant domain+peptidic linker+variable heavy chain domain+CH1+Hinge+CH2+CH3 with knob mutation)
      heavy chain (variable heavy chain domain+CH1+Hinge+CH2+CH3 with hole mutation)):
a two-armed single chain antibody
   (i.e. an antibody comprising a first binding site that specifically binds to a first epitope or antigen and a second binding site that specifically binds to a second epitope or antigen, whereby the individual chains are as follows
      combined light/heavy chain 1 (variable light chain domain+light chain constant domain+peptidic linker+variable heavy chain domain+CH1+Hinge+CH2+CH3 with hole mutation)
      combined light/heavy chain 2 (variable light chain domain+light chain constant domain+peptidic linker+variable heavy chain domain+CH1+Hinge+CH2+CH3 with knob mutation));
a common light chain bispecific antibody
   (i.e. an antibody comprising a first binding site that specifically binds to a first epitope or antigen and a second binding site that specifically binds to a second epitope or antigen, whereby the individual chains are as follows
      light chain (variable light chain domain+light chain constant domain)
      heavy chain 1 (variable heavy chain domain+CH1+Hinge+CH2+CH3 with hole mutation)
      heavy chain 2 (variable heavy chain domain+CH1+Hinge+CH2+CH3 with knob mutation)):
a T-cell bispecific antibody (TCB)
   (i.e. a full-length antibody with additional heavy chain N-terminal binding site with domain exchange comprising
      a first and a second Fab fragment, wherein each binding site of the first and the second Fab fragment specifically bind to a first antigen,
      a third Fab fragment, wherein the binding site of the third Fab fragment specifically binds to a second antigen, and wherein the third Fab fragment comprises a domain crossover such that the variable light chain domain (VL) and the variable heavy chain domain (VH) are replaced by each other, and
      an Fc-region comprising a first Fc-region polypeptide and a second Fc-region polypeptide,
   wherein the first and the second Fab fragment each comprise a heavy chain fragment and a full-length light chain,
   wherein the C-terminus of the heavy chain fragment of the first Fab fragment is fused to the N-terminus of the first Fc-region polypeptide,
   wherein the C-terminus of the heavy chain fragment of the second Fab fragment is fused to the N-terminus of the variable light chain domain of the third Fab fragment and the C-terminus of the CH1 domain of the third Fab fragment is fused to the N-terminus of the second Fc-region polypeptide);
an antibody-multimer-fusion
   (i.e. a multimeric fusion protein comprising
   (a) an antibody heavy chain and an antibody light chain, and
   (b) a first fusion polypeptide comprising in N- to C-terminal direction a first part of a non-antibody multimeric polypeptide, an antibody heavy chain CH1 domain or an antibody light chain constant domain, an antibody hinge region, an antibody heavy chain CH2 domain and an antibody heavy chain CH3 domain, and a second fusion polypeptide comprising in N- to C-terminal direction the second part of the non-antibody multimeric polypeptide and an antibody light chain constant domain if the first polypeptide comprises an antibody heavy chain CH1 domain or an antibody heavy chain CH1 domain if the first polypeptide comprises an antibody light chain constant domain,
   wherein
   (i) the antibody heavy chain of (a) and the first fusion polypeptide of (b),
   (ii) the antibody heavy chain of (a) and the antibody light chain of (a), and (iii) the first fusion polypeptide of (b) and the second fusion polypeptide of (b) are each independently of each other covalently linked to each other by at least one disulfide bond,
   wherein
   the variable domains of the antibody heavy chain and the antibody light chain form a binding site specifically binding to an antigen).

The “knobs into holes” dimerization modules and their use in antibody engineering are described in Carter P.: Ridgway J. B. B.: Presta L. G.: Immunotechnology, Volume 2, Number 1, February 1996, pp. 73-73 (1).

The CH3 domains in the heavy chains of an antibody can be altered by the “knob-into-holes” technology, which is described in detail with several examples in e.g. WO 96/027011, Ridgway, J. B., et al., Protein Eng. 9 (1996) 617-621; and Merchant, A. M., et al., Nat. Biotechnol. 16 (1998) 677-681. In this method, the interaction surfaces of the two CH3 domains are altered to increase the heterodimerization of these two CH3 domains and thereby of the polypeptide comprising them. Each of the two CH3 domains (of the two heavy chains) can be the “knob”, while the other is the “hole”. The introduction of a disulfide bridge further stabilizes the heterodimers (Merchant, A. M., et al., Nature Biotech. 16 (1998) 677-681: Atwell, S., et al., J. Mol. Biol. 270 (1997) 26-35) and increases the yield.

The mutation T366W in the CH3 domain (of an antibody heavy chain) is denoted as “knob-mutation” or “mutation knob” and the mutations T366S, L368A, Y407V in the CH3 domain (of an antibody heavy chain) are denoted as “hole-mutations” or “mutations hole” (numbering according to Kabat EU index). An additional inter-chain disulfide bridge between the CH3 domains can also be used (Merchant, A. M., et al., Nature Biotech. 16 (1998) 677-681) e.g. by introducing a S354C mutation into the CH3 domain of the heavy chain with the “knob-mutation” (denotes as “knob-cys-mutations” or “mutations knob-cys”) and by introducing a Y349C mutation into the CH3 domain of the heavy chain with the “hole-mutations” (denotes as “hole-cys-mutations” or “mutations hole-cys”) (numbering according to Kabat EU index).

The term “domain crossover” as used herein denotes that in a pair of an antibody heavy chain VH-CH1 fragment and its corresponding cognate antibody light chain, i.e. in an antibody Fab (fragment antigen binding), the domain sequence deviates from the sequence in a native antibody in that at least one heavy chain domain is substituted by its corresponding light chain domain and vice versa. There are three general types of domain crossovers, (i) the crossover of the CH1 and the CL domains, which leads by the domain crossover in the light chain to a VL-CH1 domain sequence and by the domain crossover in the heavy chain fragment to a VH-CL domain sequence (or a full length antibody heavy chain with a VH-CL-hinge-CH2-CH3 domain sequence), (ii) the domain crossover of the VH and the VL domains, which leads by the domain crossover in the light chain to a VH-CL domain sequence and by the domain crossover in the heavy chain fragment to a VL-CH1 domain sequence, and (iii) the domain crossover of the complete light chain (VL-CL) and the complete VH-CH1 heavy chain fragment (“Fab crossover”), which leads to by domain crossover to a light chain with a VH-CH1 domain sequence and by domain crossover to a heavy chain fragment with a VL-CL domain sequence (all aforementioned domain sequences are indicated in N-terminal to C-terminal direction).

As used herein the term “replaced by each other” with respect to corresponding heavy and light chain domains refers to the aforementioned domain crossovers. As such, when CH1 and CL domains are “replaced by each other” it is referred to the domain crossover mentioned under item (i) and the resulting heavy and light chain domain sequence. Accordingly, when VH and VL are “replaced by each other” it is referred to the domain crossover mentioned under item (ii); and when the CH1 and CL domains are “replaced by each other” and the VH and VL domains are “replaced by each other” it is referred to the domain crossover mentioned under item (iii). Bispecific antibodies including domain crossovers are reported, e.g. in WO 2009/080251, WO 2009/080252, WO 2009/080253, WO 2009/080254 and Schaefer, W., et al, Proc. Natl. Acad. Sci. USA 108 (2011) 11187-11192. Such antibodies are generally termed CrossMab.

In certain embodiments of all aspects and embodiments of the current invention, the multispecific antibody comprises at least one Fab fragment including either a domain crossover of the CH1 and the CL domains, or a domain crossover of the VH and the VL domains, or a domain crossover of the VH-CH1 and the VL-VL domains. In multispecific antibodies with domain crossover, the Fabs specifically binding to the same antigen(s) are constructed to be of the same domain sequence. Hence, in case more than one Fab with a domain crossover is contained in the multispecific antibody, said Fab(s) specifically bind to the same antigen.

A “humanized” antibody refers to an antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., the CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “recombinant antibody”, as used herein, denotes all antibodies (chimeric, humanized and human) that are prepared, expressed, created or isolated by recombinant means, such as using a cell according to the current invention. This includes antibodies isolated from recombinant cells such as NS0, HEK, BHK, amniocytes, or CHO cells modified according to the current invention.

As used herein, the term “antibody fragment” refers to a molecule other than an intact antibody that comprises a portion of an intact antibody and that binds to the same epitope on the same antigen to which the intact antibody binds, i.e. it is a functional fragment. Examples of antibody fragments include but are not limited to Fv: Fab: Fab′: Fab-SH: F(ab)2: bispecific Fab: diabodies: linear antibodies: single-chain antibody molecules (e.g., scFv or scFab).

### Embodiments of the Methods According to the Current Invention

Here, scifAI, a machine learning framework for the efficient and explainable analysis of high-throughput imaging data based on a modular implementation is reported.

The methods according to the current invention have been shown herein to have the potential for (i) the prediction of immunologically relevant cell class frequencies, (ii) the systematic morphological profiling of the immunological synapse, (iii) the investigation of inter-donor and inter and intra-experiment variability, as well as (iv) the characterization of the mode of action of therapeutic antibodies and (v) the prediction of their functionality in vitro.

The current invention is based, at least in part, on the finding that combining high-throughput imaging of the immunological synapse using IFC with specific data preprocessing and machine learning allows to screen for novel antibody candidates and to improve the evaluation of lead molecules in terms of functionality, mode-of-action insights and antibody characteristics such as affinity, avidity and format.

The invention is exemplified in the following using specific antibodies and techniques. This is presented solely as an example of the working of the current invention and shall not be construed as a limitation. The true scope of the invention is set forth in the appended claims.

### Comprehensive Multi-Channel Imaging Flow Cytometry Data Set of the Immunological Synapse

Using high-throughput IFC a comprehensive data set for the systematic analysis of the immunological synapse of T-cell/B-cell-conjugates (T-B conjugates) has been generated (See FIGS. 1 and 2). Human memory CD4+ T-cells, isolated from peripheral blood of different donors, were co-cultured with superantigen (Staphylococcus aureus enterotoxin A, SEA)-pulsed EBV-transformed lymphoblastoid B cells (B-LCL) expressing high levels of the co-stimulatory molecules CD86 and CD80 or left untreated (FIGS. 3-6). P-CD3zeta (Y142) as a readout of early T-cell activation, the highest titrated concentration of SEA (100 ng/mL), and a time point of 45 min was chosen to investigate functional immune synapses (FIGS. 7 and 8). In total, nine donors in four independent experiments were screened (FIG. 2) and 1,182,782 images were acquired (+SEA, FIG. 3).

It has been found that a suitable multi-channel panel for the analysis and classification consisted of brightfield (BF), F-actin (cytoskeleton), MHCII, CD3, and P-CD3zeta (TCR signaling). This allowed capturing a wide range of biologically motivated characteristics of the immunological synapse (FIG. 1).

Dead, deformed, unfocused or cropped cells were removed using a multi-step pipeline (see Examples).

Additionally, a set of 5221 images from seven randomly selected donors was labeled by an expert immunologist into nine classes organized in two levels. (FIGS. 3 and 9). The first level represented the number of existing cells in the image: singlets (n=1), doublets (n=2), and multiplets (n>2). The second level characterizes the type of the cells, their interactions to each other and the presence of TCR signaling. The singlets are composed of “single B-LCL”, “single T-cell signaling” and “single T-cell with signaling” classes. “Without” is denoted as “w/o” and “with” is denoted as “w/” in the following. The doublets include the “T-cell w/small B-LCL”, “B-LCL and T-cell in one layer”, “synapse w/o signaling”, “synapse w/signaling”, and “no cell-cell interaction” classes. The class “multi-synapse”, contains more than two cells and at least one B-LCL and T-cell.

Without being bound by this theory, it is assumed that the “T-cell w/small B-LCL” and “no cell-cell interaction” classes were artifacts of the experiments. However, they were annotated to enhance the predictive power of classification models and subsequently filtered out and not used in further analyses (see Examples).

### ScifAI: An Explainable AI Framework for the Analysis of Multi-Channel Imaging Flow Cytometry Data

Herein is reported a single-cell imaging flow cytometry AI (scifAI) module.

Universally applicable for single-cell imaging projects, the module provides functionality for import and preprocessing of input data, several feature engineering pipelines including the implementation of a set of biologically motivated features and autoencoder-generated features (see Examples), as well as methodology for efficient and meaningful feature selection.

Moreover, the module implements several machine learning and deep learning models for training supervised image classification models, e.g. for the prediction of cell configurations such as the immunological synapse. Following the principle of multi-instance learning, the module also implements functionality to regress a set of selected images, against a downstream continuous readout such as cytokine production.

Extensive documentation, as well as example code in the form of Jupyter notebooks is provided online at https://github.com/marrlab/scifAI/ and https://github.com/marrlab/scifAI-notebooks.

### Scifai for High-Throughput Profiling of the Immunological Synapse

In order to characterize the immunological synapse in an unbiased fashion, first a series of biologically motivated, interpretable features using the scifAI module has been designed and computed. These features were based on morphology, intensity, co-localization, texture and synaptic features extracted from the 5-panel stained images and their corresponding masks (see Examples and FIGS. 10-11). Synaptic features were implemented based on the ratio of the signal intensity of each fluorescent channel in the synaptic area to the whole cell. Leveraging the large amount of unlabeled data, a multi-channel autoencoder to learn a second set of data-driven features from the images in an unsupervised fashion was implemented [24]. The autoencoder was designed to encode the images to a 128-dimensional abstract feature space by reconstructing the input images (see Examples).

Subsequently, scifAI was used to compose a supervised machine learning pipeline for the classification of the 5221 annotated images across the nine immunologically relevant cell classes. A series of supervised machine learning models for the prediction of all nine classes using both the interpretable feature space as well as the abstract autoencoder features across all donors and experimental conditions was trained and benchmarked. The models included an XGBoost classifier on the interpretable features and a multi-class logistic regression (LR) on the interpretable and data-driven features. To pre-select the features and reduce the dimensionality, a feature pre-selection pipeline using an ensemble of different methods was implemented (see Examples and FIG. 12-13). For comparison a number of convolutional neural network (CNN) architectures such as Resnet18, ResNet34, DeseNet121 and DeepFlow which had previously been shown to be successful in classification tasks on imaging flow cytometry data was trained [22,24,28]. The CNN architectures intrinsically learned a feature representation based on the input images and their corresponding labels. All models were trained on a stratified subset, comprising 2923 (70%) annotated images. In order to benchmark the classification model and feature space combinations, macro F1 scores on the remainder of images as the hold-out test set comprising 1567 (30%) annotated images was compared (see Examples). The XGBoost model using the interpretable feature set performed best (F1−macro=0.93±0.01, mean±std bootstrapping with n=1000) among all the classifiers.

Thus, in certain embodiments of all aspects and embodiments of the methods according to the current invention, an XGBoost model using an interpretable feature set is used.

The XGBoost model was followed by convolutional neural networks ResNet34 (0.92±0.01), ResNet18 (0.91±0.01), DeepFlow (0.90±0.01), DenseNet121 (0.90±0.02), the multi-class logistic regression using the interpretable feature set (0.89±0.02), and logistic regression using the data-driven feature set (0.83±0.02).

It has been found that the XGBoost model provides for the best compromise between performance and explainability. Thus, the XGBoost model was selected as the final classifier for label expansion to the full dataset (FIG. 4).

Investigation of the model's confusion matrix on the hold-out set revealed that misclassifications occurred mostly within the cell classes' signaling property, whereas all other classes showed good overall concordance (see FIG. 14).

After training the XGBoost classifier, it has been explored which underlying features drive the class prediction. Therefore, the features have been ranked by their respective Gini-index (see FIG. 5). The most predictive features were based on colocalization of CD3 & MHCII, colocalization of MHCII & P-CD3zeta, texture of MHCII and CD3, and intensity of P-CD3zeta.

In certain embodiments of all aspects and embodiments of the methods according to the invention the doublets and multiplets of cells are classified in addition to be a synapse based on one or more of the following
the (correlated) distance of the MHCII and the CD3 labelling,
the distance of the centers of the MHCII and CD3 labelling,
the Manders overlap coefficient of MHCII and P-CD3zeta labelling,
the homogeneity of the CD3 labelling,
the contrast of the MHCII labelling,
the Kurtosis intensity of the P-CD3zeta labelling,
the Manders overlap coefficient of the MHCII and CD3 labelling, or/and
the maximum intensity of the CD3 labelling.

In one preferred embodiment of all aspects and embodiments of the methods according to the invention the doublets and multiplets of cells are classified in addition to be a synapse based on one or more of the following
the colocalization of the CD3 and MHCII labelling, or/and
the colocalization of the MHCII and P-CD3zeta labelling, or/and
the texture of the MHCII, or/and
the texture of the CD3 labelling, or/and
the intensity of the P-CD3zeta labelling.

Without being bound by this theory, it is assumed that based on the features and the definition of classes (i) the texture of the CD3 and MHCII labelling can be used to detect the existence of T- and B-LCL cells in an image, (ii) the colocalization of the CD3 and MHCII labelling can be used to detect the different doublets types and (iii) the intensity of the P-CD3zeta labelling and the colocalization of the MHCII and P-CD3zeta labelling can be used to detect whether it is a signaling T-cell (see FIG. 15).

### A Subset of Annotated Data and Available IFC Channels is Sufficient for a High Classification Performance

It has been further investigated how many annotated samples were necessary to reach a reasonable classification performance. Therefore, the model was repeatedly trained using stratified subsets of the training data and evaluated the F1-macro on the test set. The results showed that by using 1500 images (45% of the training data), 90% of F1-macro on the test set could be achieved (FIG. 16).

Additionally it has been determined, which channels were sufficient for reaching high performance. Therefore, the BF channel was kept and all possible combinations of the fluorescent channels were used to train the model.

It has been found that the channels BF, MHCII and P-CD3zeta are sufficient to reach an F1-macro similar to using all the channels (FIG. 17).

### Characterizing the Impact of Therapeutic Antibodies on Synapse Formation

It has been further investigated the effect of therapeutic antibodies on the formation of the immunological synapse and to better characterize their morphological profiles. This analysis included the investigation of potential class frequency changes and feature differences.

Two antibodies have been used for the investigation: one activator and one inhibitor of immune responses. The activating T-cell bi-specific (TCB) antibody was designed to target CD3 and CD19, a co-receptor of B cells [29] (see FIG. 18). The inhibitory antibody, Teplizumab, is described to only bind to CD3 (see FIG. 19) and has been shown to dampen T cell responses [30,31]. For each antibody an appropriate control (Ctrl-TCB and isotype, respectively) was run within the same experiment and donor. Since Teplizumab required an existing immune response for subsequent inhibition, SEA was used to first stimulate the T-cells (see FIG. 18). The same setup was also used for the isotype control. Six donors across two experiments for CD19-TCB and seven donors across three experiments for Teplizumab were measured (see FIGS. 20-23). To determine class frequency changes between the antibody and its control, the previous XGBoost classifier (see FIGS. 4 and 15) was used to predict the class for all images based on the interpretable features (see Examples and FIG. 24). To ensure that the previously trained XGBoost model was transferable from +SEA to the antibody experiments, an expert annotated a randomly selected subset of 396 images for CD19-TCB and 227 images for Teplizumab. A high concordance between the expert annotations and the XGBoost predictions on the new experiments (macro F1-score=0.86 for TCB and 0.85 for the Teplizumab) confirmed that the trained model is generalizable and can thus be utilized for further analyses (see FIG. 25).

For a compact representation of class frequency changes, a log 2-fold change value was calculated between the antibodies and their respective controls.

Focusing on the feature differences of synapses under antibody stimulation the images predicted as “synapses w/signaling” for each donor were selected and the interpretable features from only fluorescent channels including texture, synaptic features, morphology, intensity and co-localization between antibodies and their controls were compared. The BF channel was not included as its intensity is difficult to interpret and its morphological characteristics is captured by other fluorescent channels (see Methods).

In certain embodiments of all aspects and embodiments according to the current invention, the method is for determining class frequency change in the presence of a therapeutic antibody, wherein number or frequency of doublets and multiplets of synapses with signaling in the absence and the presence of the therapeutic antibody is determined, or/and wherein the interpretable features from the fluorescent channels including texture, synaptic features, morphology, intensity and co-localization between antibodies and their controls were compared.

### Cd19-TCB Increases the Formation of Stable Immune Synapses

Stimulation of the immune response by CD19-TCB led to a significant increase of doublets and multiplets frequencies. The “synapse w/signaling” class showed thereby the highest increase (median log_2 (CD19-TCB/Ctrl-TCB)=2.6, n=6 donors, p=0.036) followed by “multiplet of two B-cells and one T-cell forming a synapse with signaling” (median=1.94, p=0.036), “B-LCL & T-cell in one layer” (median=1.77, p=0.036), and “doublet or multiplet of one or two B-cells and one T-cell forming a synapse without signaling” class (median=0.44, p=0.036). For the singlets, the overall trend was a decrease in class frequency of “single B-LCL” (median=−0.29, p=0.036) and “single T-cell w/o signaling” (median=−0.78, p=0.036) (see FIG. 26).

Additionally, the feature differences in synapses induced by the CD19-TCB was analyzed (see Methods), comparing the 210 interpretable features from all fluorescent channels. It has been found that out of 210*6=1260 possibilities combinations of features and donors, 210 features were significantly increased and 163 features were significantly decreased (see FIG. 27). All donors exhibited mostly similar responses towards the stimulation with CD19-TCB. On average 27±4 features were significantly decreased and 33±7 features were significantly increased per donor (dashed lines bottom FIG. 27). From these features, a number of features with similar changes within at least 4 out of 6 donors have been identified (FIG. 27 and Table 1).
Table 1: Significant features induced by CD19-TCB. Shown are the features that significantly changed for at least four donors after addition of CD19-TCB. The table represents the list of consistent features from FIG. 27. 1 represents a significant increase (red in FIG. 27), −1 represents a significant decrease (blue in FIGS. 27) and 0 represents no significant change (gray in FIG. 27).

| | | do- | do- | do- | do- | do- | do- |
|---|---|---|---|---|---|---|---|
| | | nor | nor | nor | nor | nor | nor |
| feature | category | 7 | 8 | 1 | 5 | 2 | 9 |
| homogeneity_P-CD3zeta | texture | 1 | 0 | 1 | 0 | 1 | 1 |
| dissimilarity_P-CD3zeta | texture | −1 | −1 | −1 | 0 | −1 | −1 |
| mean_intensity_ratio_P- | synaptic | 1 | 0 | 1 | 1 | 1 | 1 |
| CD3zeta_R5_R7 | feature | | | | | | |
| mean_intensity_ratio_P- | synaptic | 1 | 0 | 1 | 1 | 1 | 1 |
| CD3zeta_R5_R6 | feature | | | | | | |
| mean_intensity_ratio_CD | synaptic | 1 | 0 | 1 | 1 | 1 | 1 |
| 3_R5_R7 | feature | | | | | | |
| sum_intensity_ratio_CD3 | synaptic | 1 | 0 | 1 | 1 | 1 | 1 |
| R5_R7 | feature | | | | | | |
| mean_intensity_ratio_CD | synaptic | 1 | 0 | 1 | 1 | 1 | 1 |
| 3_R5_R6 | feature | | | | | | |
| sum_intensity_ratio_CD3_ | synaptic | 1 | 0 | 1 | 1 | 1 | 1 |
| R5_R6 | feature | | | | | | |
| sum_intensity_ratio_MH | synaptic | 1 | 0 | 1 | 1 | 1 | 1 |
| CII_R5_R7 | feature | | | | | | |
| sum_intensity_ratio_P- | synaptic | 1 | 0 | 1 | 1 | 1 | 1 |
| CD3zeta_R5_R7 | feature | | | | | | |
| sum_intensity_ratio_P- | synaptic | 1 | 0 | 1 | 1 | 1 | 1 |
| CD3zeta_R5_R6 | feature | | | | | | |
| mean_intensity_ratio_M | synaptic | 1 | 0 | 1 | 1 | 1 | 1 |
| HCII_R5_R6 | feature | | | | | | |
| max_intensity_ratio_P- | synaptic | 1 | 1 | 1 | 0 | 1 | 0 |
| CD3zeta_R5_R6 | feature | | | | | | |
| max_intensity_ratio_P- | synaptic | 1 | 1 | 1 | 0 | 1 | 0 |
| CD3zeta_R5_R7 | feature | | | | | | |
| mean_intensity_ratio_M | synaptic | 1 | 0 | 1 | 1 | 1 | 1 |
| HCII_R5_R7 | feature | | | | | | |
| sum_intensity_ratio_MH | synaptic | 1 | 0 | 1 | 1 | 1 | 1 |
| CII_R5_R6 | feature | | | | | | |
| mask_based_eccentricity_ | morphol- | 1 | 1 | 1 | 0 | 1 | 1 |
| P-CD3zeta | ogy | | | | | | |
| mask_based_eccentricity_ | morphol- | 1 | 1 | 1 | 0 | 1 | 1 |
| CD3 | ogy | | | | | | |
| mask_based_solidity_P- | morphol- | −1 | −1 | −1 | −1 | −1 | 0 |
| CD3zeta | ogy | | | | | | |
| mask_based_solidity_M | morphol- | −1 | −1 | −1 | −1 | −1 | 0 |
| HCII | ogy | | | | | | |
| mask_based_extent_P- | morphol- | 0 | −1 | −1 | −1 | −1 | 0 |
| CD3zeta | ogy | | | | | | |
| mask_based_extent_CD3 | morphol- | 0 | −1 | −1 | −1 | −1 | 0 |
| | ogy | | | | | | |
| mask_based_extent_MH | morphol- | −1 | 0 | −1 | −1 | −1 | 0 |
| CII | ogy | | | | | | |
| mask_based_solidity_CD | morphol- | −1 | −1 | −1 | −1 | −1 | 0 |
| 3 | ogy | | | | | | |
| sum_intensity_P- | intensity | 0 | 1 | 1 | 0 | 1 | 1 |
| CD3zeta | statistics | | | | | | |
| mean_intensity_P- | intensity | 1 | 1 | 1 | 1 | 1 | 1 |
| CD3zeta | statistics | | | | | | |
| skew_intensity_P- | intensity | 1 | 1 | 1 | 1 | 1 | 1 |
| CD3zeta | statistics | | | | | | |
| max_intensity_P- | intensity | 1 | 1 | 1 | 1 | 1 | 1 |
| CD3zeta | statistics | | | | | | |
| std_intensity_P-CD3zeta | intensity | 1 | 1 | 1 | 1 | 1 | −1 |
| | statistics | | | | | | |
| kurtosis_intensity_P- | intensity | 1 | 1 | 1 | 1 | 1 | 1 |
| CD3zeta | statistics | | | | | | |
| shannon_entropy_P- | intensity | 1 | 1 | 1 | 1 | 1 | −1 |
| CD3zeta | statistics | | | | | | |
| dice_distance_MHCII_P- | colocali- | −1 | 0 | −1 | −1 | −1 | −1 |
| CD3zeta | zation | | | | | | |
| correlation_distance_R6_ | colocali- | −1 | −1 | −1 | −1 | −1 | −1 |
| CD3_R7_P-CD3zeta | zation | | | | | | |
| correlation_distance_R4_ | colocali- | −1 | −1 | 0 | 0 | −1 | −1 |
| F-Actin_R5_MHCII | zation | | | | | | |
| euclidean_distance_R7_P- | colocali- | −1 | −1 | 0 | −1 | 0 | −1 |
| CD3zeta_R6_CD3 | zation | | | | | | |
| euclidean_distance_R6_C | colocali- | −1 | −1 | 0 | −1 | 0 | −1 |
| D3_R7_P-CD3zeta | zation | | | | | | |
| correlation_distance_R7_ | colocali- | −1 | −1 | −1 | −1 | −1 | −1 |
| P-CD3zeta_R6_CD3 | zation | | | | | | |
| jaccard_distance_MHCII_ | colocali- | −1 | 0 | −1 | −1 | −1 | −1 |
| P-CD3zeta | zation | | | | | | |
| correlation_distance_R6_ | colocali- | −1 | 0 | −1 | −1 | −1 | −1 |
| CD3_R5_MHCII | zation | | | | | | |
| correlation_distance_R5_ | colocali- | −1 | 0 | −1 | −1 | −1 | −1 |
| MHCII_R6_CD3 | zation | | | | | | |
| jaccard_distance_MHCII_ | colocali- | −1 | 0 | −1 | −1 | −1 | −1 |
| CD3 | zation | | | | | | |
| dice_distance_MHCII_C | colocali- | −1 | 0 | −1 | −1 | −1 | −1 |
| D3 | zation | | | | | | |
| correlation_distance_R7_ | colocali- | −1 | 0 | −1 | −1 | −1 | −1 |
| P-CD3zeta_R5_MHCII | zation | | | | | | |
| correlation_distance_R5_ | colocali- | −1 | 0 | −1 | −1 | −1 | −1 |
| MHCII_R7_P-CD3zeta | zation | | | | | | |
| correlation_distance_R5_ | colocali- | −1 | −1 | 0 | 0 | −1 | −1 |
| MHCII_R4_F-Actin | zation | | | | | | |

Additionally, an increase in “mean intensity of P-CD3zeta” has been identified, similar to SEA stimulation, with higher enrichment within the synaptic area (see FIGS. 28-29 and Table 1).

Furthermore, a stronger enrichment of F-actin and MHCII towards the synapse has been found (FIGS. 31-34).

Thus, the addition of the therapeutic antibody resulted in an increase in the frequencies of doublets and multiplets as well as a (stronger) enrichment of F-actin and MHCII in the synaptic area. Without behind bound by theory, it is assumed that this is indicative of an enhanced formation of tight immunological synapses, translating into an efficient TCR signaling. These observations are in line with the mode of action that has already described in general for TCBs, promoting a stable interaction between tumor cells and T-cells [32-33].

### Teplizumab Alters Synapse Formation and TCR Signaling

In contrast to the CD19-TCB, the presence of Teplizumab reduced the frequency of doublets and multiplets significantly (FIG. 35). The highest decrease was observed for the “doublet of one B-cell and one T-cell forming a synapse with signaling” class (median log_2 (Teplizumab/Isotype)=−0.75, n=7, p=0.018), followed by “multiplet of two B-cells and one T-cell forming a synapse with signaling” (median=−0.51, p=0.031), “a doublet of a B-cell and a T-cell forming a synapse without signaling” (median=−0.44, p=0.018), and “B-LCL & T-cell in one layer” (median=−0.18, p=0.018). Accordingly, “single T-cell w/signaling” (median=0.66, p=0.018) and “single B-LCL” (median=0.07, p=0.018) were increased significantly as compared to the isotype. Surprisingly, the “T-cell w/o signaling” class frequency was significantly decreased (median=−0.35, p=0.018), probably due to the significant increase of “single T-cell w/signaling” (see FIG. 35).

Feature differences in synapses induced by Teplizumab in seven donors was analyzed. From “a doublet of a B-cell and a T-cell forming a synapse without signaling” images 132 features based on F-actin, MHCII and P-CD3zeta and their co-localizations were extracted. CD3 features could not be included for the analysis as the binding of Teplizumab and the anti-CD3 staining antibody interfere. Therefore, an anti-CD4 staining antibody was used to identify T-cells. 131 significantly increased and 169 significantly decreased features out of 132*7=924 possibilities were identified (FIG. 30 and Table 2).
Table 2: Significant features induced by Teplizwab. Shown are the features that significantly changed for at least six donors after addition of Teplizumab. The table represents the list of consistent features from FIG. 30. 1 represents a significant increase (red in FIG. 30), −1 represents a significant decrease (blue in FIG. 30) and 0 represents no significant change (gray in FIG. 30).

| | | do- | do- | do- | do- | do- | do- | do- |
|---|---|---|---|---|---|---|---|---|
| | | nor | nor | nor | nor | nor | nor | nor |
| feature | category | 6 | 1 (1) | 2 | 1 (2) | 5 | 3 | 4 |
| homogeneity_F-Actin | texture | −1 | −1 | 1 | −1 | −1 | −1 | 1 |
| energy_F-Actin | texture | −1 | 0 | 1 | −1 | −1 | −1 | 1 |
| ASM_F-Actin | texture | −1 | 0 | 1 | −1 | −1 | −1 | 1 |
| energy_P-CD3zeta | texture | 0 | −1 | −1 | −1 | 1 | 1 | −1 |
| ASM_P-CD3zeta | texture | 0 | −1 | −1 | −1 | 1 | 1 | −1 |
| max_intensity_ratio_P- | synaptic | −1 | −1 | 0 | −1 | −1 | −1 | 0 |
| CD3zeta_R5_R7 | feature | | | | | | | |
| sum_intensity_F-Actin | intensity | −1 | −1 | 1 | −1 | −1 | −1 | 1 |
| | statistics | | | | | | | |
| min_intensity_P-CD3zeta | intensity | 1 | 1 | −1 | 1 | 0 | 1 | 0 |
| | statistics | | | | | | | |
| sum_intensity_P-CD3zeta | intensity | 1 | 0 | −1 | 1 | 0 | 1 | 1 |
| | statistics | | | | | | | |
| skew_intensity_P-CD3zeta | intensity | 0 | 0 | −1 | −1 | −1 | 0 | −1 |
| | statistics | | | | | | | |
| shannon_entropy_F-Actin | intensity | −1 | −1 | 1 | −1 | −1 | −1 | 1 |
| | statistics | | | | | | | |
| max_intensity_F-Actin | intensity | −1 | −1 | 1 | −1 | −1 | −1 | 1 |
| | statistics | | | | | | | |
| min_intensity_F-Actin | intensity | −1 | −1 | 1 | −1 | −1 | −1 | 1 |
| | statistics | | | | | | | |
| std_intensity_F-Actin | intensity | −1 | −1 | 1 | −1 | −1 | −1 | 1 |
| | statistics | | | | | | | |
| mean_intensity_F-Actin | intensity | −1 | −1 | 1 | −1 | −1 | −1 | 1 |
| | statistics | | | | | | | |
| euclidean_distance_R7_P- | colocali- | −1 | −1 | 1 | −1 | −1 | −1 | 1 |
| CD3zeta_R4_F-Actin | zation | | | | | | | |
| euclidean_distance_R4_F- | colocali- | −1 | −1 | 1 | −1 | −1 | −1 | 1 |
| Actin_R7_P-CD3zeta | zation | | | | | | | |
| manders_overlap_ | colocali- | 1 | 1 | −1 | −1 | 1 | 1 | −1 |
| coefficient_ | zation | | | | | | | |
| R5_MHCII_R4_F-Actin | | | | | | | | |
| manders_overlap_ | colocali- | 1 | 1 | −1 | 1 | 1 | 1 | −1 |
| coefficient_R7_ | zation | | | | | | | |
| P-CD3zeta_R4_F-Actin | | | | | | | | |
| manders_overlap_ | colocali- | 1 | 1 | −1 | 1 | 1 | 1 | −1 |
| coefficient_R4_F-Actin_ | zation | | | | | | | |
| R7_P-CD3zeta | | | | | | | | |
| structural_similarity_R5_ | colocali- | −1 | −1 | 1 | 0 | −1 | −1 | 1 |
| MHCII_R7_P-CD3zeta | zation | | | | | | | |
| structural_similarity_R7_P- | colocali- | −1 | −1 | 1 | 0 | −1 | −1 | 1 |
| CD3zeta_R5_MHCII | zation | | | | | | | |
| manders_overlap_ | colocali- | 1 | 1 | −1 | −1 | 1 | 1 | −1 |
| coefficient_R4_F-Actin_ | zation | | | | | | | |
| R5_MHCII | | | | | | | | |

In particular, Teplizumab, on average, led to 25±8 significantly decreased features and 19±17 significantly increased features per donor (dashed lines bottom of FIG. 30). Donor 6 showed the least number of changes with five significantly increased features. In contrast, donor 4 showed the highest number of increased features with 50 features. A set of features, which were significantly increased or decreased for at least 5 out 7 donors was identified (FIGS. 36-37). A decrease in the mean intensity of F-actin was identified whereas donor 2 and 4 indicated a significant increase (FIGS. 36 and 38). This opposite reaction of the two donors could be also detected for other F-actin related features (Table 2). Besides the changes in F-actin features, also a significant reduction of P-CD3zeta intensity within the synapse was detected and a stronger clustering of TCR signaling around the whole T-cell was observed (FIGS. 37 and 39).

Thus, with the method according to the current invention new insights into the immunosuppressive mode of action of Teplizumab was obtained as a reduction in the number of synapses as well as changes in the F-actin reorganization and P-CD3zeta signaling towards the synapse were identified.

### CONCLUSIONS

With the method according to the current invention, the mode of action of therapeutic antibodies can be analyzed.

Additionally, with the method according to the current invention it is possible to predict the functionality of therapeutic antibodies in vitro.

The current invention is based, at least in part, on the generation of morphological profiles of the immunological synapse that allowed characterizing the mode of action of therapeutic antibodies early after the initiation of an immune response. Thereby a prediction of the related downstream T-cell responses can be done.

These findings and methods are different to previous works that did not take into account inter-experiments effects on synapse formation [21,35].

The current invention is based, at least in part, on the detection and determination of changes of immunological synapses. This has been achieved, at least in part, by the inclusion of interpretable feature extracted from the fluorescence images in a machine learning framework. Thereby it has been achieved that the method according to the current invention is scalable, provides reproducible results and facilitates the deployment into existing workflows, which differs from previous works that use a combinations for each stage of the analysis [27,36,37].

Without being bound by this theory, it is assumed that the combination of interpretable features and explainable machine learning allowed for the identification of relevant morphological classes, such as immunological synapses, with the same or even better accuracy as state-of-the-art methods. Thereby it was possible to analyze the morphological profiles of the immunological synapse in an unbiased way as well as the characterization of the mode of action of antibodies in a biologically relevant context.

Thus, the methods according to the current invention are an improvement compared to known methods that are primarily focused on performance over interpretability [26,38].

The capabilities of the methods according to the invention have been shown by analyzing the effects of two therapeutic antibodies on the immunological synapse, the CD19-TCB and Teplizumab, which are both binding CD3 and have been described to activate and suppress T-cell responses, respectively [29-31].

It has been found with the method according to the current invention that in the presence of CD19-TCB more stable immune synapses are formed, which is indicated by a stronger enrichment of MHCII and F-actin within the synapse that was paralleled by a higher intensity of P-CD3zeta labelling.

It has been found with the method according to the invention that in the presence of Teplizumab a decrease in synapse formation and prevention of F-actin reorganization as well as localization of P-CD3zeta towards the synapse occurs.

These observations give new insights into the immunosuppressive mode of action of Teplizumab that has rarely been investigated in vitro so far [30,31].

Without being bound by this theory, it can be assumed that the reduced P-CD3zeta intensity in the synaptic area and the observed non-polarized distribution of P-CD3zeta signal around the whole T-cell could indicate altered TCR signaling that might be translated into a reduced T-cell effector function. High numbers of peripheral P-CD3zeta microclusters have been reported for self-reactive T-cells with altered synapse formation and aberrant T-cell responses [6].

Unexpectedly, the methods according to the invention allowed for the identification of features within the synapse class revealing inter donor-variability upon stimulation with the different antibodies.

Thus, the methods according to the current invention enable the rapid screening for responders in vitro and pre-select suitable patients for clinical trials.

Taken together, by applying the methods according to the current invention it was possible to thoroughly investigate the mode of action of therapeutic antibodies based on significant features and also to gain more insights into inter donor-variability that might potentially translate into different functional outcomes in vivo.

With the methods according to the current invention, the state-of-the-art methods have been improved by incorporating biologically motivated features such as texture, intensity statistics and synaptic related features.

For the first time interpretable features have been used for the immunological synapse to predict the effectiveness of therapeutic antibodies on T-cell cytokine production. Thereby it became possible to predict the functional outcome of an unseen antibody and to pinpoint the driving factors required for the prediction.

For example, for a TCBs it has been found that intensity of MHCII and morphology of F-actin are the most prominent features in predicting cytokine readouts.

The ability to predict unseen antibodies allows the investigation of various antibody formats to better understand mechanistically how different formats can impact T-cell responses and help to guide format selection.

The methods according to the invention encompass data acquisition and analysis that can be adjusted to investigate various hypotheses and to develop diverse applications based on imaging flow cytometry data.

For instance, while in the current examples memory CD4+ T-cells were analyzed as they are poised to show faster immune responses and a higher synapse propensity compared to naive T-cells [49], imaging and analysis of CD8+ T cells, as the main players in cytotoxicity, can likewise be done to similarly elaborate how synapse features correlate with killing efficiency of therapeutic antibodies against tumor cells.

The methods according to the current invention can also be utilized in the design of IFC experiments, optimizing the number and type of stainings, as well as the total number of images per donor to be acquired.

The methods according to the current invention can be used to improve the quality and the speed of antibody development, for example giving new insights towards the mode of action of particular candidate molecules, or to predict in vitro efficacy in high-throughput. The identification of lead molecules and better prioritization in terms of epitope, affinity, avidity and antibody format will have a huge impact on the decision making process.

Above that, the methods according to the current invention can even help to identify responders among patient populations and predict their clinical outcomes.

All references cited herein are expressly incorporated by reference in their entirety.

The following examples and figures are provided to aid the understanding of the present invention, the true scope of which is set forth in the appended claims. It is understood that modifications can be made in the procedures set forth without departing from the spirit of the invention.

### DESCRIPTION OF THE FIGURES

FIG. 1 Schematic representation of the data generation and analysis pipeline. To systematically analyze the immunological synapse of T-B-cell conjugates, 1,182,782 images were acquired with an imaging flow cytometer. After that, these can be manually classified (right) or scifAI (left) can be used to extract morphological features, train machine learning models, profile immunological synapses and characterize the functionality of therapeutic antibodies.

FIG. 2 Gating strategy to identify single interacting T-B-LCL synapses using the IDEAS software of the imaging flow cytometer.

FIG. 3 A subset of 5221 images was manually annotated by an expert into nine immunologically relevant classes that can be grouped into singlets (either B- or T-cells), doublets (with one B- and one T-cell), and multiplets (containing more than 2 cells). Cell images show brightfield (BF, scale bar=2.4 μm), F-actin (cytoskeleton), MHCII, CD3 and P-CD3ζ (P-CD3zeta) (a marker for TCR signaling).

FIG. 4 Six different approaches to train predictive machine learning models for the identification of the immunologically relevant classes were benchmarked, combining different classification algorithms and feature engineering strategies. These approaches included interpretable (interp.) features combined with explainable classifiers, an autoencoder to generate data-driven features, an explainable classifier, and three convolutional neural networks. Interpretable features combined with the XGBoost classifier resulted in the best trade-off between interpretability and classification performance.

FIG. 5 List of the donors, their age, gender and experiment numbers that are used in this study.

FIG. 6 List of experiments and donors with and without SEA.

FIG. 7 Testing of assay conditions using conventional FACS. Primary memory CD4+ T-cells isolated from PBMCs of healthy donors were stimulated with B-LCL cells in the presence of different concentrations of SEA (0.1-100 ng/mL) or left untreated (−SEA). Frequencies of P-CD3ζ+ (P-CD3zeta positive), TNF-α+ (TNFalpha positive) and CD69+ CD4+ (CD69 and CD4 positive) T-cells were determined at various time points. The small FACS histograms in the bar graphs show the expression levels of the three markers by comparing the highest concentration of SEA (100 ng/mL) with the untreated control (−SEA) after 60 min. The data shown represents one experiment using T-cells from three different donors.

FIG. 8 Percentage of single T-B-LCL synapses and P-CD3ζ+ CD4+ (P−CD3zeta and CD4 positive) T-cells measured by imaging flow cytometry between two different SEA concentrations (10 and 100 ng/mL) after 45 and 120 min. Data represents two donors.

FIG. 9 Number of labeled by expert data per donor.

FIG. 10 Visual representation of each multi-channel image and corresponding masks. Masks were exported along with images from the IDEAS software.

FIG. 11 List of interpretable features. The morphology, intensity statistics, textures synaptic features are based on one channel. The colocalization features are based on two channels. ScifAI automatically detects the existing channels and generates the specified features.

FIG. 12 Feature pre-selection pipeline to reduce the dimensionality of the feature space and removing multicollinearity. First the highly correlated features were dropped. Then an ensemble of different classifiers was trained on the data, and their top-k features were selected. Finally, hierarchical clustering was done on top of the union of the features to account for multicollinearity.

FIG. 13 The number of selected features before passing the selected features to the XGBoost classifier. For obtaining the best top-k the data selection pipeline+XGBoost was trained on stratified randomly selected 85% of the training set and tested on the rest 15%.

FIG. 14 Confusion matrix of the data selection pipeline (top-k=211)+XGBoost, based on the predictions on the test set.

FIG. 15 Top eight features for the detection of cell classes were ranked based on Gini-index. The features include colocalization, texture and intensity of MHCII, CD3 and P-CD3ζ (P-CD3zeta). The exemplary images are taken from donor 7 sampling from the 5th, 50th and 95th percentile of the distribution of each feature.

FIG. 16 Number of annotated images vs. the classification performance.

FIG. 17 The XGBoost model was used to train the classifier. The training data was used for this evaluation using a 5-fold cross validation. In each step, features based on the selected channels were used for training the classifier. As brightfield (BF) is a stain-free channel, it is always kept in the data. The combinations are ranked based F1-macro.

FIG. 18 Schematic representation of the mode of action of Teplizumab.

FIG. 19 Schematic representation of the mode of action of CD19-TCB.

FIG. 20 Donors and their respective experiments that were used for the class frequency analysis and feature difference analysis.

FIG. 21 Donors and their respective experiments that were used for the class frequency analysis and feature difference analysis.

FIG. 22 List of experiments and donors with TCBs and their control.

FIG. 23 List of experiments and donors with Teplizumab and isotype control.

FIG. 24 Frequency of classes for the main classes as explained in Methods. Each dot represents a donor, color coded by the experiments.

FIG. 25 Confusion matrix for classifications in CD19-TCB and Teplizumab based on 396 and 227 expert-annotated images, respectively. The previously trained model (FIG. 4) reached a macro F1-score of 0.86 and 0.85, respectively, on both datasets.

FIG. 26 Class frequency differences depicted as log 2 fold-changes between CD19-TCB and its corresponding control (Ctrl TCB). Each dot represents a donor color coded as in FIG. 21. The vertical black line is the median across donors for each class.

FIG. 27 Systematic comparison of 210 relevant features between CD19-TCB and Ctrl-TCB across images predicted as “synapse w/signaling” across six donors. Each line represents a feature and each column represents a donor. For each donor, the features that are significantly increased are depicted with dark grey/black and the significantly decreased ones are depicted with light grey. The donors are sorted based on the number of significantly changed features. The bottom bar plot shows the count of increased or decreased features per donor.

FIG. 28 Statistical and visual inter-donor comparison of the representative feature “mean intensity P-CD3zeta” between CD19-TCB and Ctrl-TCB. For visualization purposes, the features are mapped between zero and one for each donor separately.

FIG. 29 Visual representative for the representative feature “mean intensity P-CD3zeta” which were randomly sampled for both Ctrl-TCB and CD19-TCB from donor 9, and were found to be in concordance with the statistical results (scale bar=2.4 μm).

FIG. 30 Systematic comparison of 132 relevant features between Teplizumab and isotype across images predicted as “synapse w/signaling” among all six donors. Each line represents a feature and each column represents a donor. For each donor, the features that are significantly increased are depicted with dark grey/black and the significantly decreased ones are depicted with light grey. The donors are sorted based on the number of significantly changed features. The bottom bar plot shows the count of increased or decreased features per donor.

FIG. 31 Statistical and visual inter-donor comparison of the representative feature “F-actin enrichment in synapse” between CD19-TCB and Ctrl-TCB. For visualization purposes, the features are mapped between zero and one for each donor separately.

FIG. 32 Visual representative for the representative feature “F-actin enrichment in synapse” which were randomly sampled for both Ctrl-TCB and CD19-TCB from donor 9, and were found to be in concordance with the statistical results (scale bar=2.4 μm).

FIG. 33 Statistical and visual inter-donor comparison of the representative feature “MHCII enrichment in synapse” between CD19-TCB and Ctrl-TCB. For visualization purposes, the features are mapped between zero and one for each donor separately.

FIG. 34 Visual representative for the representative feature “MHCII enrichment in synapse” which were randomly sampled for both Ctrl-TCB and CD19-TCB from donor 9, and were found to be in concordance with the statistical results (scale bar=2.4 μm).

FIG. 35 Class frequency differences depicted as log 2 fold-changes between Teplizumab and its corresponding control (isotype). Each dot represents a donor color coded as in FIG. 22. The vertical black line is the median across donors for each class.

FIG. 36 Statistical and visual inter-donor comparison of the feature F-actin between Teplizumab and its isotype.

FIG. 37 Statistical and visual inter-donor comparison of the feature P-CD3zeta between Teplizumab and its isotype.

FIG. 38 Visual representatives for the feature F-actin were randomly sampled for both isotype and Teplizumab from donor 3 and were found to be in concordance with the statistical results (scale bar=2.4 μm).

FIG. 39 Visual representatives for the feature P-CD3zeta were randomly sampled for both isotype and Teplizumab from donor 3 and were found to be in concordance with the statistical results (scale bar=2.4 μm).

FIG. 40 Algorithmic flowchart of the method for classifying cells in a cell mixture using single-cell imaging flow cytometry in combination with artificial intelligence (scifAI) according to the current invention.

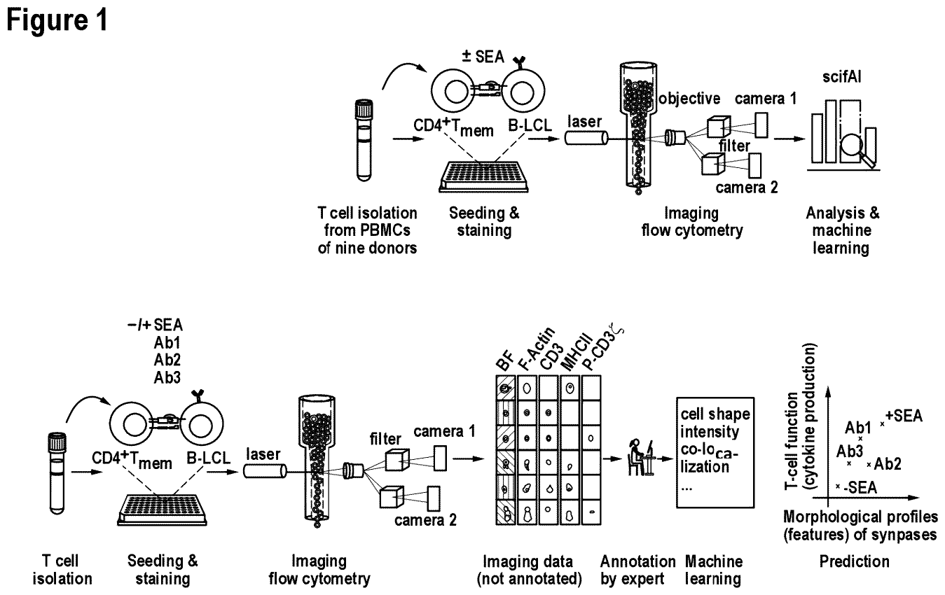

### MATERIALS AND METHODS

### Cell Culture

EBV-transformed B-lymphoblastoid cell line (B-LCL) from donor 333 was obtained from Astarte Biologics (#1038-3161JN16) and cells were cultivated in RPMI-1640 medium (PAN-Biotech: cat #P04-17500) with 10% FBS (Anprotec: cat #AC-SM-0014Hi) and 2 mM L-glutamine (PAN-Biotech: cat #P04-80100). Z138 (MCL, gift from University of Leicester) and Nalm-6 (ALL, DSMZ ACC 128) tumor cells were cultivated in RPMI1640 containing 10% FBS and 1% Glutamax (Invitrogen/Gibco #35050-038).

### Immune Synapse Formation and Imaging Flow Cytometry

To analyze immune synapses, human memory CD4+ T cells were isolated from PBMCs of nine healthy human donors using a negative selection EasySep Enrichment kit from STEMCELL Technologies (cat #19157). Live/dead staining of T- and B-LCL-cells was separately performed using the fixable viability dye eF780 for 15 min at RT (eBioscience: cat #65-0865-14). Cells were then re-suspended in RPMI-1640 medium supplemented with 10% FBS (Anprotec: cat #AC-SM-0014Hi), 5% Penicillin-Streptomycin (Gibco: cat #15140-122) and 2 mM L-glutamine (PAN-Biotech: cat #P04-80100). Afterwards B-LCL-cells were transferred into a well of a 96-well round bottom plate (300,000 cells per well) and were pre-incubated with the superantigen Staphylococcal enterotoxin A (SEA) (Sigma-Aldrich: cat #S9399) for 15 min at 37° C. or left untreated. Human CD4+Tₘₑₘ-cells were added to the afore-prepared B-LCL-cells (250,000 cells per well) to generate a final ratio of 4:3 (B-LCL: Tₘₑₘ) and subsequently the appropriate in-house made compounds (10 μg/mL of Isotype Ctrl or Teplizumab and 1 μg/mL (5 nM) of Ctrl-TCB or CD19-TCB) were added to the B-LCL-Tₘₑₘ-cell co-culture. To strengthen the conjugate formation between B-LCL- and T-cells they were centrifuged at 300×g for 30 sec and then directly transferred to a 37° C. incubator for 45 min. Thereafter, the medium in each well was carefully aspirated with a pipette and cells were immediately fixed for 12 min at RT followed by permeabilization using the Foxp3/Transcription factor staining buffer set from eBioscience (cat #00-5523-00).

Intracellular staining was performed in permeabilization buffer containing fluorescently-labeled antibodies for 40 min at 4° C.: CD3-BV421 (clone UCHT1, Biolegend: cat #300433), HLA-DR-PE-Cy7 (clone L243, Biolegend: cat #307616), Phalloidin AF594 (ThermoFisher: cat #A12381) and P-CD3ζ Y142-AF647 (K25-407.69, BD cat #558489).

After washing, cells were suspended in FACS buffer (PBS supplemented with 2% FBS) and acquired on an Amnis ImageStreamX Mark II Imaging Flow Cytometer (Luminex) equipped with five lasers (405, 488, 561, 592 and 640 nm). On average, around 55,000 images were collected per sample at 60× magnification on a low speed setting. IDEAS software (version 6.2.187.0, EMD Millipore) was used for data analysis and labeling of cells.

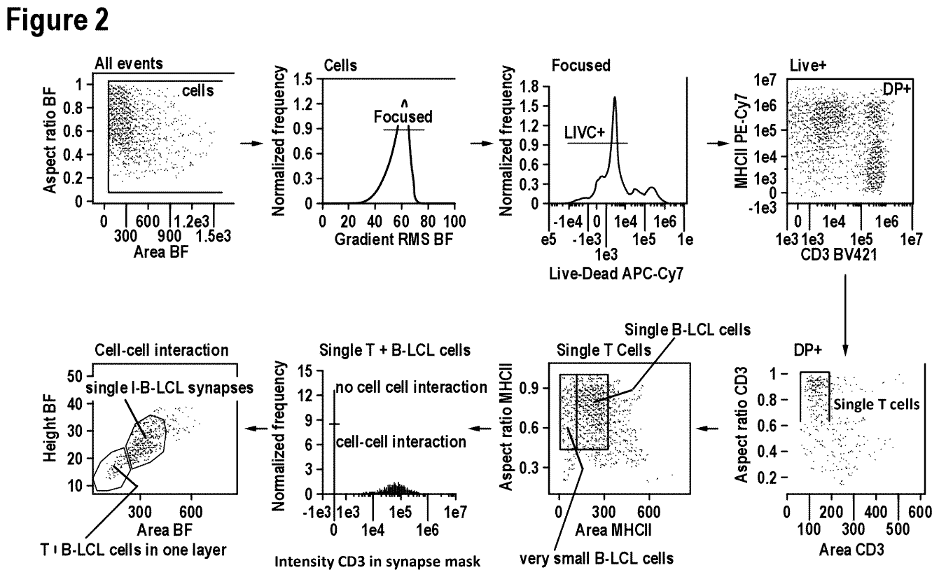

To identify immune synapses using the IDEAS software the gating strategy in FIG. 2 was implemented. Cells were first gated on in-focus live+ CD3+ MHCII+ cells. Within this population, images that show single CD3+ T cells and single MHCII+ B-LCL cells were selected using the area and aspect ratio feature. Next, to exclude non-interacting cells the CD3 intensity within a self-created synapse mask was determined. The term “mask” as used herein denotes the outer silhouette of the overlay of all images (BF and all labelled antibodies) obtained for a cell or doublet or multiplet, respectively. The synapse mask was defined as a combination of the morphology CD3 and MHCII mask with a dilation of 3. Only synapses that showed a CD3 signal in the mask were gated. Finally, T+ B-LCL cells in one layer were excluded by using the height and area feature of the brightfield (BF) and single T-B-LCL synapses were analyzed.

### Intracellular Staining of Cytokines Using Conventional Flow Cytometry

For intracellular cytokine staining cells were first treated with GolgiPlug (BD Biosciences: cat #555029) and GolgiStop (BD Biosciences: cat #554724) for at least 2-4 h before being stained. After incubation live/dead staining was performed using the fixable viability dye eF780 for 20 min at 4° C. (eBioscience: cat #65-0865-14). Cells were then fixed and permeabilized using the Foxp3/Transcription factor staining buffer set from eBioscience (cat #00-5523-00) as described for the synapse formation assay. Intracellular staining was performed in permeabilization buffer containing fluorescently-labeled antibodies for 30 min at 4° C.: TNFα-APC (clone MAb11, BD Biosciences: cat #554514), IFN-γ-PE (clone B27, BD Biosciences: cat #554701) and Granzyme B-PE-Cy7 (clone QA16A02, Biolegend: cat #372214). Finally, cells were suspended in FACS buffer (PBS supplemented with 2% FBS and 1 mM EDTA) and acquired on a FACS Celesta from BD Biosciences.

### Tumor Cell Lysis Assays (In Vitro)

B-cell-depleted PBMCs derived from blood of healthy donors were prepared using standard density-gradient isolation followed by B cell depletion with CD20 Microbeads (Miltenyi: cat #130-091-104). B cell-depleted PBMCs were then incubated with the tumor targets (Z-138 or Nalm-6) at a ratio of 5:1 for 24 h in the presence or absence of CD19-TCB. Tumor cell lysis was calculated based on LDH release (LDH Cytotoxicity Detection Kit from Roche Applied Science) and normalized to spontaneous release (PBMCs+targets without treatment=0% tumor cell lysis) and maximal release (lysis of tumor targets with Triton X-100=100% lysis).

### Quantification of CD19 Expression

CD19 expression on B-LCL-cells were determined using the Quantum™ Alexa Fluor® 647 MESF Kit from Bangs Laboratories (cat #647) according to the manufacturer's instructions using an anti-human CD19-AF647 (Biolegend #302220) antibody as well as the corresponding isotype control muIgG2b (Biolegend #400330). For the quantification of CD19 molecules on the tumor target cell lines Nalm-6 and Z-138 the QiFi Kit from Dako (cat #K0078) was performed according to the manufacturer's instructions by using an anti-human CD19 purified (BD #555410) antibody as well as the corresponding isotype control muIgG2b (BD #557351).

### Preparation of the Imaging Dataset for Analysis

In total 2,899,575 imaging flow cytometry images were recorded. The dataset consists of nine distinct donors across four independent experiments. Donor 1 and Donor 2 were used twice. Different conditions were measured which included-SEA (total images=625,001), +SEA (557,781), Ctrl-TCB (330,000), CD19-TCB (324,020), Isotype (405,000), and Teplizumab (403,375). The images contained brightfield (BF), F-actin, MHCII, CD3, P-CD3ζ, and live-dead stainings. The live-dead staining is only used to filter out the dead cells. For each experiment, the images were compensated using a compensation matrix derived from stained single cells. After the compensation, the raw images (16-bit) and their corresponding channel-wise segmentation masks were exported from the IDEAS software and saved in an HDF5 format. To enable parallelization, each image and its corresponding mask were saved separately.

Interpretable Feature Engineering from Images

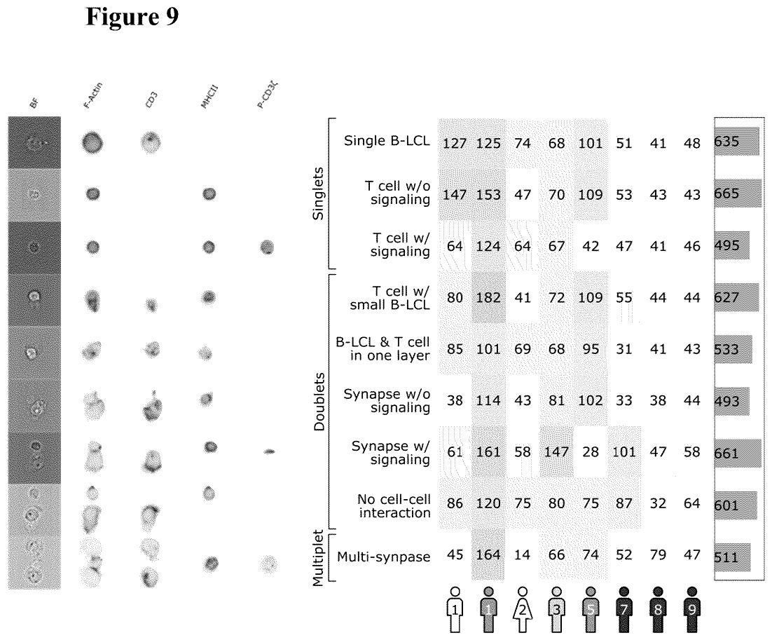

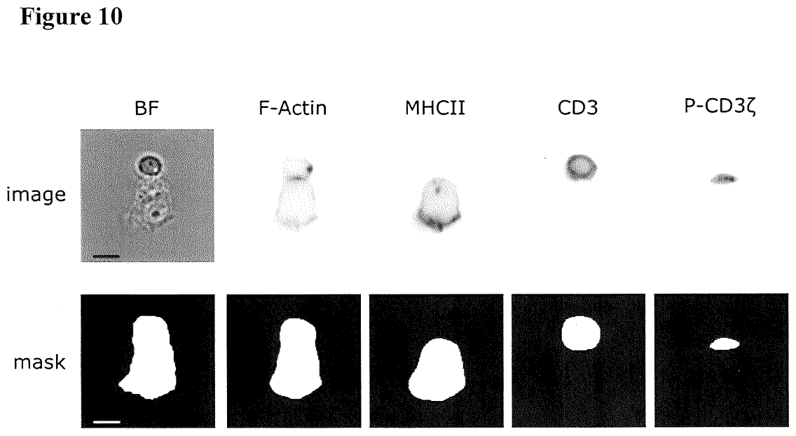

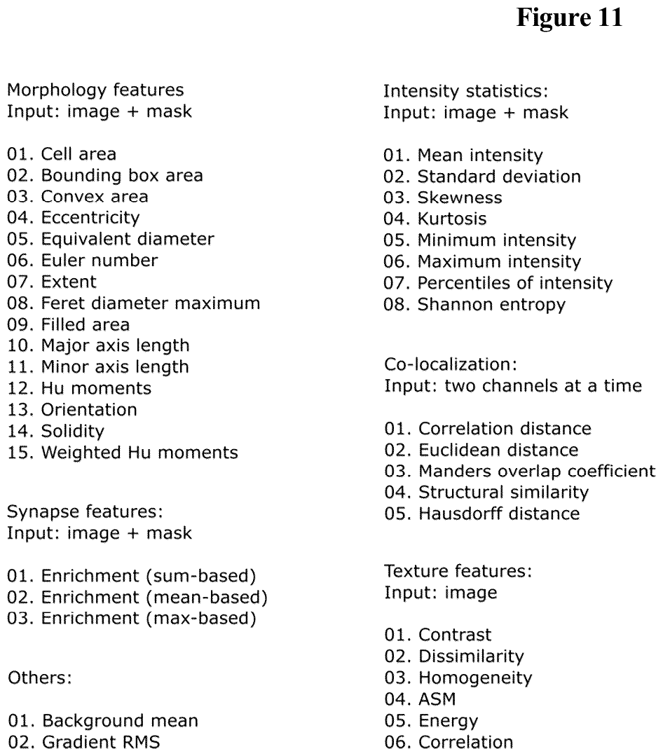

A set of 296 biologically motivated features was extracted to study the immunological synapse. These features included morphology, intensity, co-localization, texture and synaptic related values (see FIGS. 5, 6, 9, 22, 32). The morphology features were calculated based on the segmentation mask from each channel. The features included “area”, “bounding box area”, “convex area”, “eccentricity”, “equivalent diameter”, “Euler number”, “extent”, “maximum Feret diameter”, “minimum Feret diameter”, “filled area”, “length of major axis”, “length of minor axis”, “Hu moments”, “orientation”, “perimeter”, “Crofton perimeter”, “solidity”, “weighted Hu moments”. All the morphology features are extracted using scikit-image library [41]. For the intensity features, first the cells were segmented using their corresponding mask. The intensity features included “min”, “sum”, “mean”, “standard deviation”, “skewness”, “kurtosis”, “max” and “Shanon entropy”. In addition, the percentile of intensity values including “10th percentile”, “20^{th} percentile”, . . . “90th percentile” were calculated. All of the intensity features were calculated based on NumPy [42] and SciPy [43] functionality. For co-localization features, a “dice distance” and “Jaccard distance” was implemented to calculate the masks overlap between two channels using the SciPy [43] library. In addition, the “correlation distance” [43], “Euclidean distance” [43], “Manders overlap coefficient” [44], “intensity correlation quotient” [44], “structural similarity” [41] and “Hausdorff distance” [41] were calculated. For texture features, we used Gray Level Co-occurrence Matrix (GLCM) features [45] including “contrast”, “dissimilarity”, “homogeneity”, “ASM”, “energy” and “correlation”. The synapse related features were defined as “enrichment of Ch (mean)”=(intensity of Ch in synapse)/m (intensity of Ch), “enrichment of Ch (sum)”=sum (intensity of Ch in synapse)/sum (intensity of Ch), and “enrichment of Ch (max)”=max(intensity of Ch in synapse)/mean(intensity of Ch) [35]. Finally, “background mean” and “gradient RMS” were implemented for quality control of images. All these features were implemented using NumPy (version=1.18.5), Pandas (1.1.5), SciPy (1.8.0), scikit-image (0.19.2), and scikit-learn (1.0.2) [46].

### Autoencoder Feature Extraction

To leverage the large amount of unlabeled data, we implemented and trained a multichannel autoencoder [24]. This autoencoder included a separate encoder for each channel. The encoders were designed to map each channel to a 32 dimensional vector. The concatenation of these vectors led to a 5*32 dimensional space. Then these features were mapped to a 128 dimensional feature vector. A decoder on top of the concatenated vectors was implemented for reconstructing the original image. “L2 norm” was used as the reconstruction loss. The augmentations used for training the autoencoder included random rotation, random scaling, random flipping, random Gaussian noise.

### Feature Pre-Selection

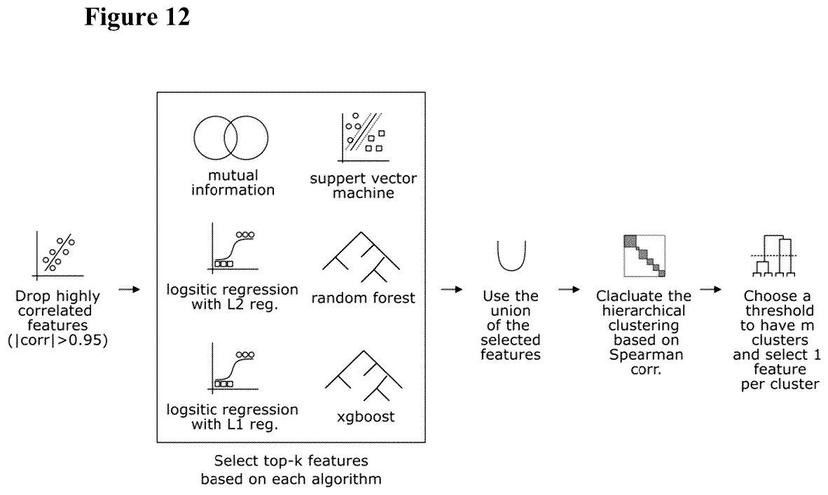

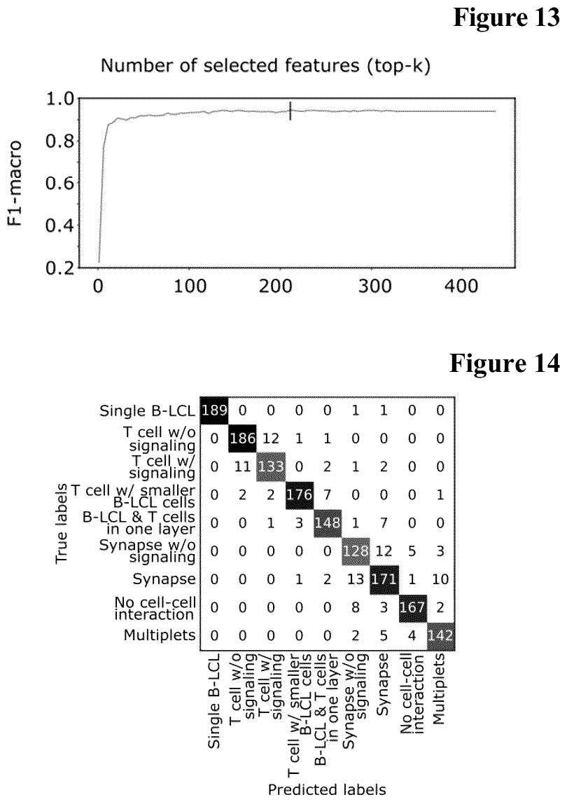

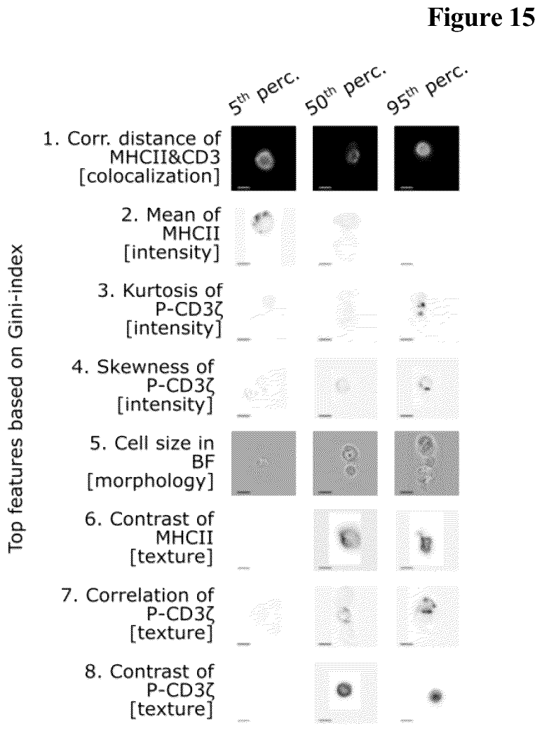

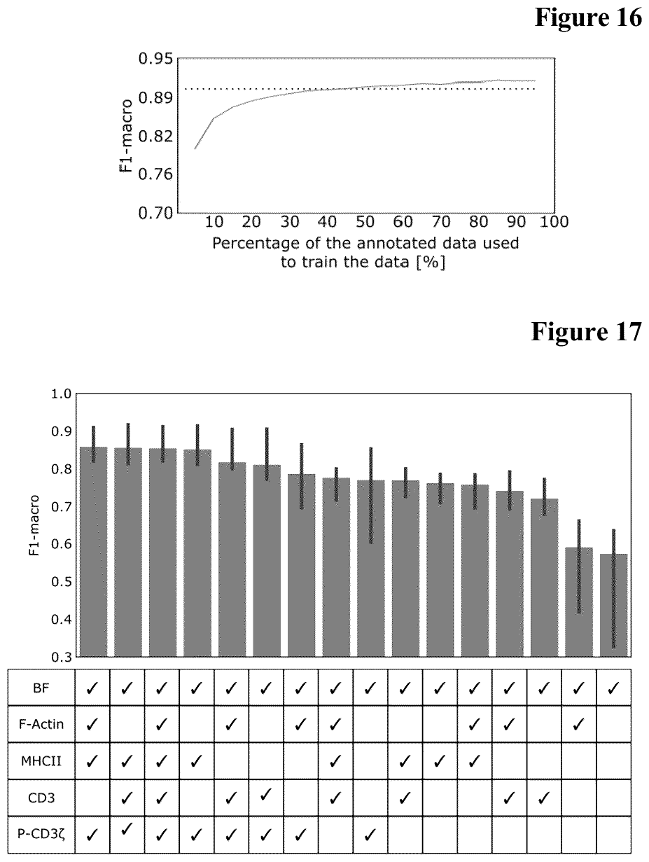

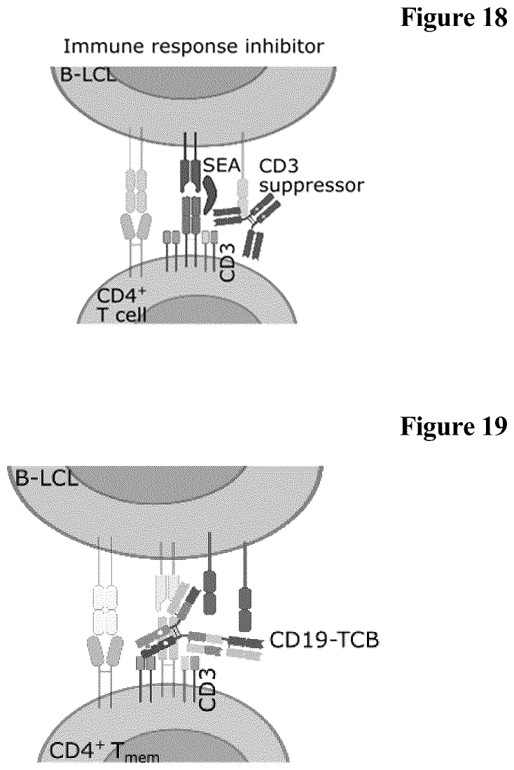

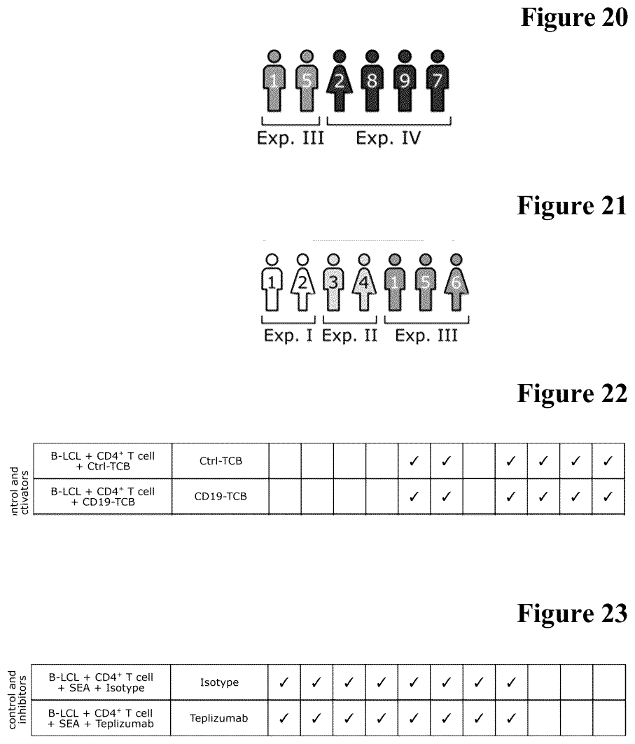

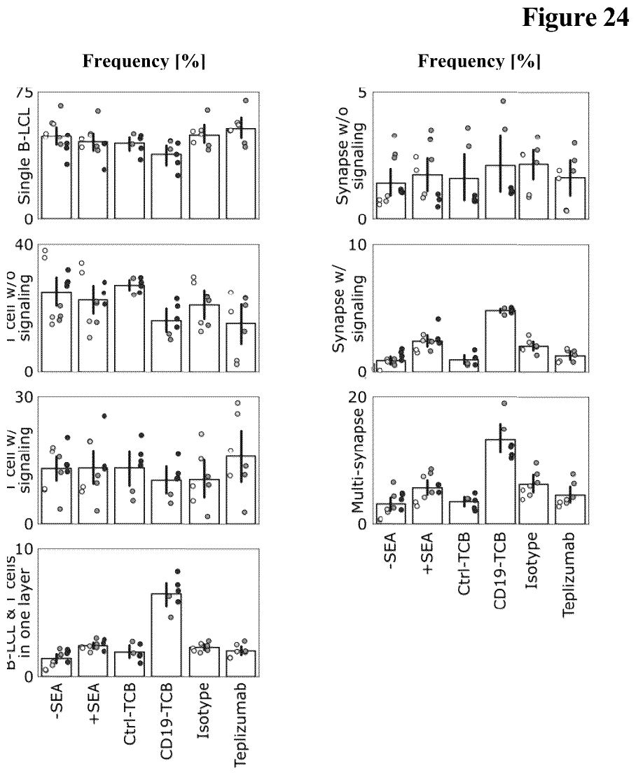

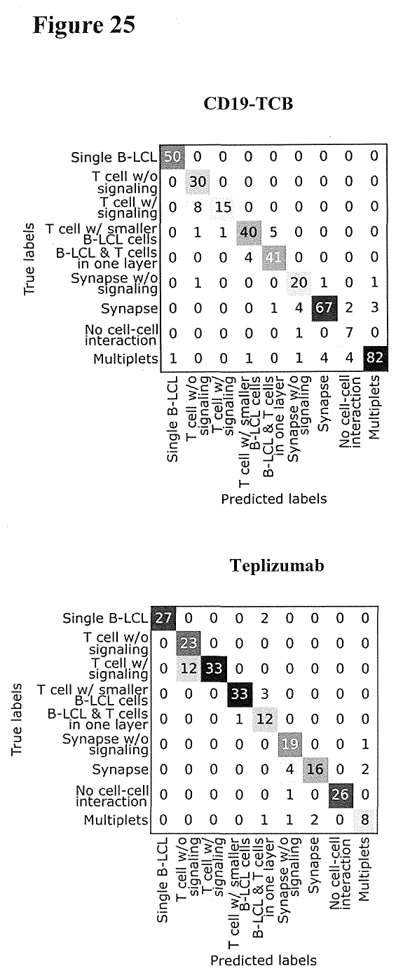

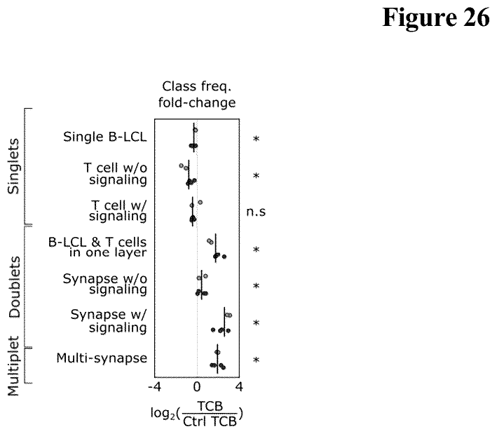

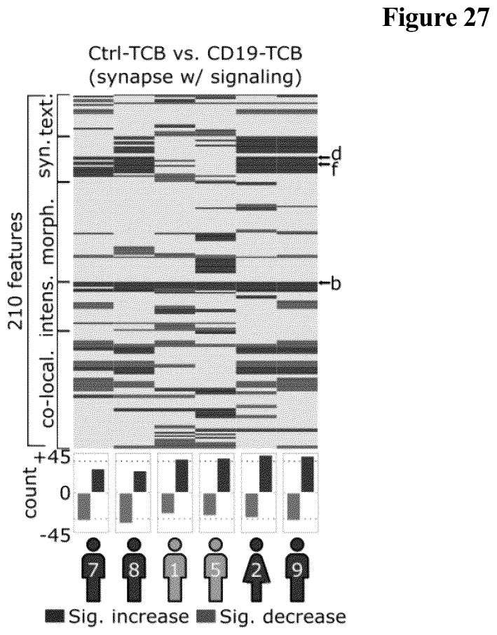

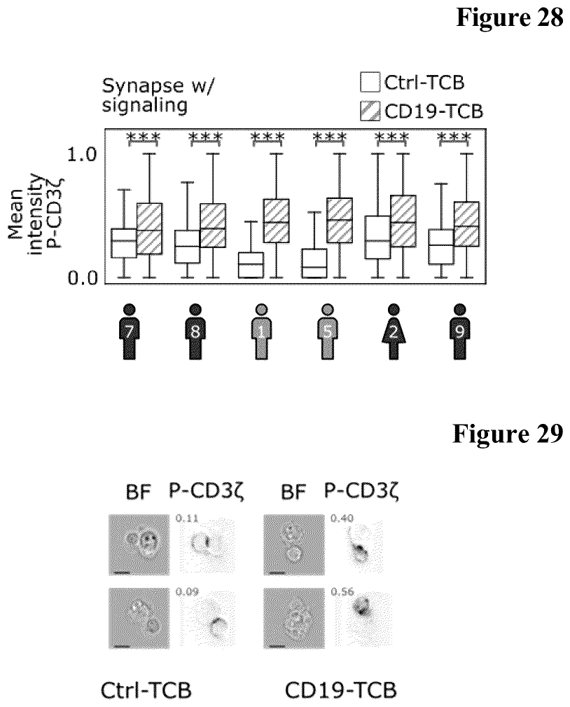

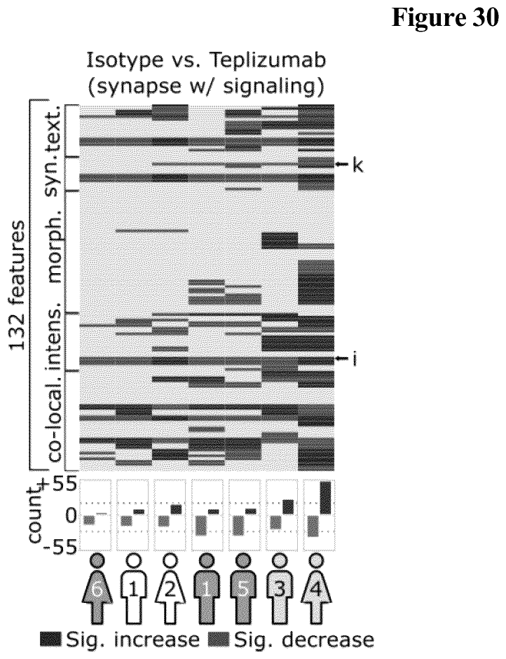

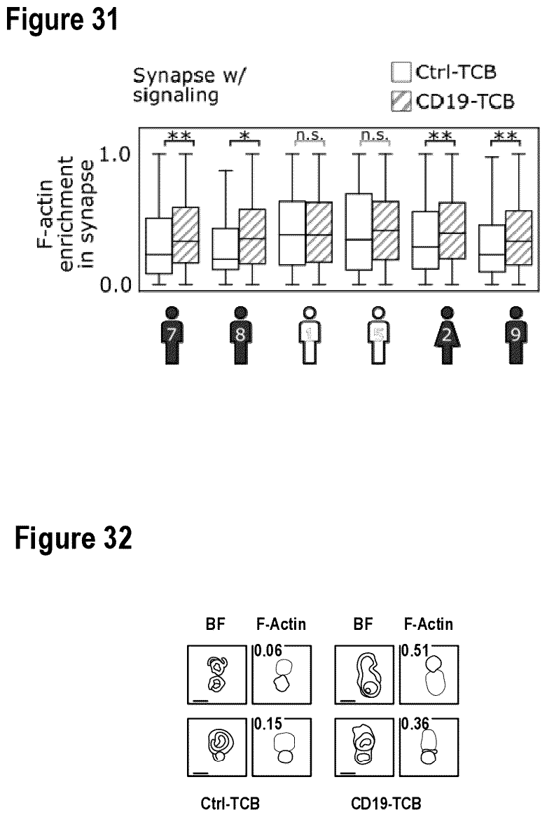

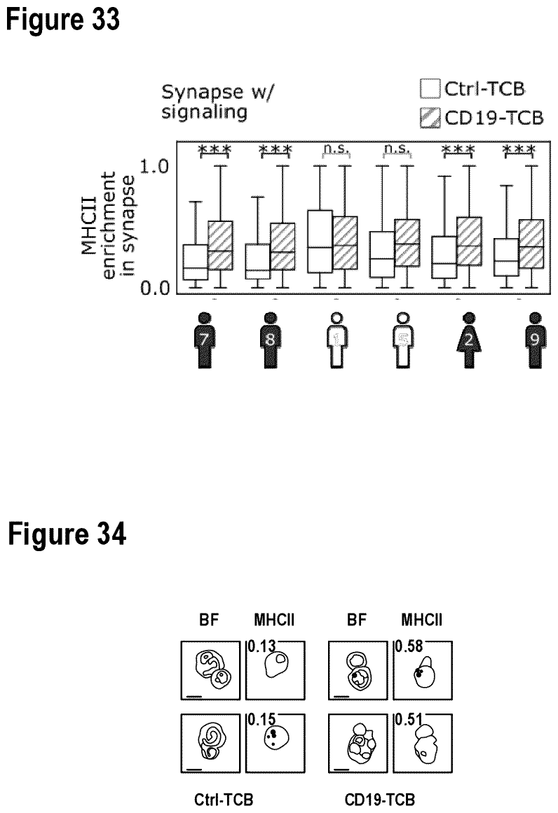

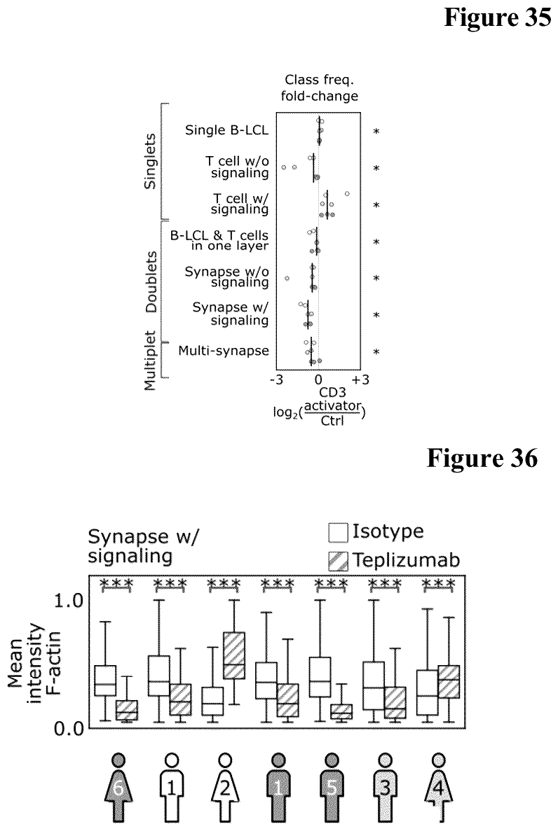

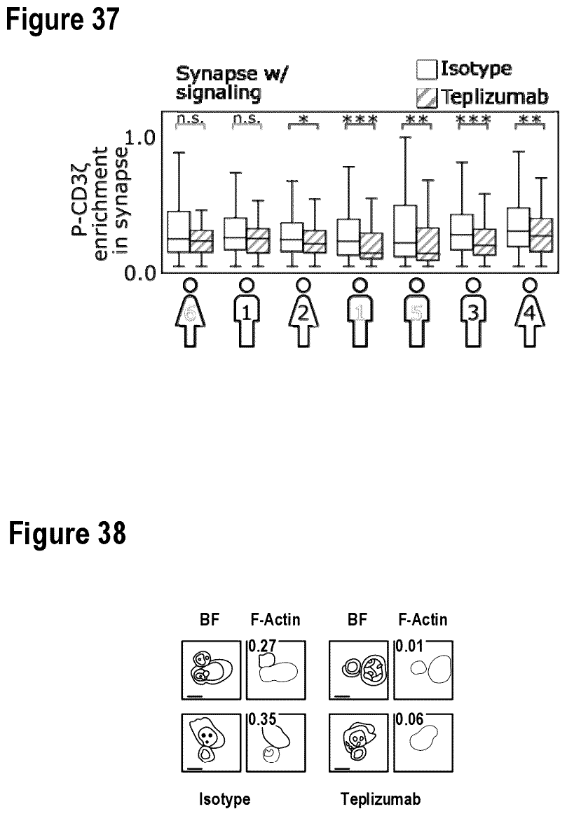

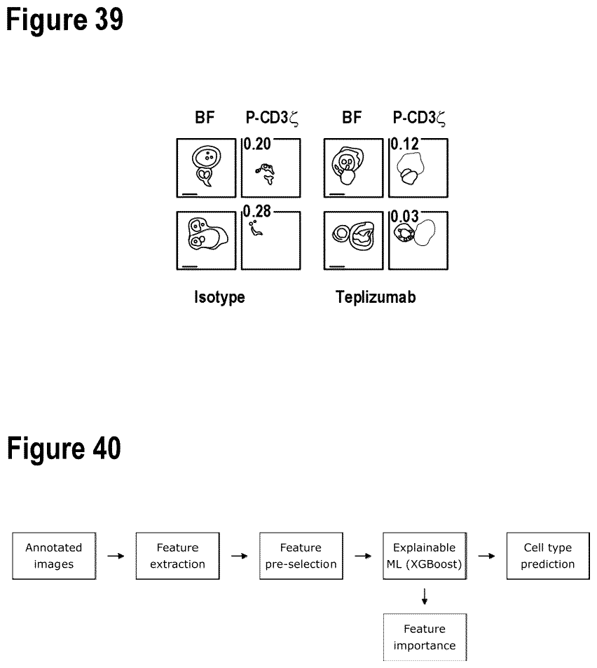

Considering that the number of features was large, a feature pre-selection pipeline was implemented to select the most relevant features according to the work of Haq et al. [47] (see FIGS. 12-14). First, the Pearson correlation between the features was measured. If at least two features were highly correlated (|corr|>0.95), then only one of them was kept (at random), and the rest were eliminated. In the next step, six different methods were used to rank the features. These methods included mutual information, linear support vector machine, logistic regression with L1 regularization, logistic regression with L2 regularization, random forest, and XGBoost. The top-k (hyper-parameter to be selected) features from each method were selected, and their union was used. After this reduction, the Spearman correlation matrix between the features was calculated, and spectral clustering was performed on the correlations. Then, m clusters were created, and one feature at random per cluster was selected. The last step was performed to account for multicollinearity between the features.

### Classification

There are three main approaches that are used for training a supervised learning algorithm, feature based approach and deep learning.

### Classical Supervised Learning Models

Two different algorithms were used for training machine learning models. A boosting method called XGBoost was used which uses an ensemble of trees on the data (n_trees=100). The second model was a logistic regression. The advantage of using these models was that they provide explainability after the training.

### Convolutional Neural Networks

For training supervised deep learning models, well-known architectures in the field of computer vision including ResNet18, Resnet34, ResNet50, ResNet152, DeseNet121 and DeepFlow were used [22,24,28]. All of the models were pre-trained on ImageNet. Considering the models are designed for three channels input, the first convolutional layer with three input channels to six input channels was removed. In addition, the classification layer also needed to be adjusted to have nine classes. However, the rest of the networks were untouched with their predefined ImageNet weights. We used multi-class cross entropy loss for training. The learning rate (lr) was set to 0.001, with adaptive strategy of reducing on plateau of 10 epochs. The augmentations used for training the autoencoder included random rotation, random scaling, random flipping, random Gaussian noise.

### Classification Feature Importance

A feature pre-selection filtering was used to reduce the number of features and then an XGBoost classifier was trained on the annotated data. While the XGBoost can provide feature importance using Gini-index, these importances can be biased due to different reasons such as correlation between the pre-selected features, number of features, the pre-selection process, outliers, etc. To account for this, the training data was spitted randomly to 5-folds (stratified) and trained the XGBoost classifier five times, each time using 4 out of the 5 folds. This process was repeated 100 times, leading to 500 different models. In each training, a random number of pre-selected features (top-k) between 30 to 200 features was used. Eventually, for every feature a series of Gini-indices were obtained. The median Gini-index for each feature was used to rank the features.

### Classification Staining Importance

To determine which staining contributes the most to the predictions, a recursive channel elimination was used. In every run, BF was kept as it is stain-free. Then train the “interpretable features+XGBoost” based on the features of the selected channels.

### Class Frequency Analysis

For each donor, first the trained XGBoost classifier was used to predict the classes for every image. Then we excluded images using this data cleaning protocol:
1. Filtering out images including dead cells (using Live-Dead staining) with “mean Live-Dead intensity”>=“mean Live-Dead intensity (90th perc.)”
2. Filtering out dead cells (using Live-Dead staining) with “mean Live-Dead intensity”> “mean Live-Dead intensity (90th perc.)”
3. Filtering out unfocused images with these conditions “Gradient RMS BF”>“Gradient RMS BF (2nd perc.)” and “Gradient RMS BF”<“Gradient RMS BF (90th perc.)”
4. Filtering out images based with high entropy using the XGBoost predictions (entropy>1.0). The entropy was calculated using SciPy package. This step is done to omit images that the classifier is the most uncertain in terms of prediction.
5. Filtering out images predicted as “B-LCL” with “mean intensity of MHCII”<“mean intensity of MHCII (5th perc.)”. This step guarantees that the images predicted “B-LCL” contain a minimum MHCII intensity
6. Filtering out images predicted as “B-LCL” with “area of MHCII”<“area of MHCII (10th perc.)”. This step guarantees that the images predicted “B-LCL” contain a cell with appropriate size and reduces artifacts.
7. Filtering out images predicted as “T-cell” with “mean intensity of CD3”< “mean intensity of CD3 (1st perc.)”. This step guarantees that the images predicted “T cell” contain a minimum CD3 intensity.
8. Filtering out images predicted as “B-LCL and T-cell in one layer” with “area of MHCII”<“area of MHCII (20th perc.)”. This step is performed to omit “B-LCL and T-cell in one layer” with small “B-LCL's”.
9. Filtering out images based on the isolation forest outlier detection. It was used n_estimators-100, max_samples=“auto”, contamination=“auto”, and max_features=20 as the main parameters. For reducing the run time, only top 30 features were used based on Gini-index from the XGBoost training.
10. Filtering out images based on Uniform Manifold Approximation and Projection (UMAP).

First, all images were ztransformed to 2D dimensional space using UMAP. The features were standardized using the mean and std of each feature. For reducing the run time, only top 30 features based on Gini-index from the XGBoost training were used. Then a DBSCAN algorithm was run with eps-0.09 and min_samples=5. The resulted clustered were filtered out if (#images in cluster)/(#total images)<0.0001.

All these steps are done based on scikit-learn implementations. All parameters were set using the default value of scikit-learn unless stated otherwise. After the data cleaning, the frequency of each class was calculated with “F_C=(#images predicted as C)/(#total images)” for each condition per donor. To deal with the compositional nature of the data, a log_2 (F_C_antibody/F_C_control) was used to compare the frequency fold-changes. This transformation has the advantage that the frequencies do not sum to a constant value. After calculating the log_2 fold-changes, the Wilcoxon-rank-sum test was used for analyzing the effects of antibodies on class frequencies. Wilcoxon-rank-sum tests whether two samples are likely to derive from the same population. To account for multiple testing, a Benjamini-Hochberg correction was used for +SEA/−SEA, CD19-TCB/Control TCB and Teplizumab/isotype respectively. Because the experiments were performed independently, only each of these comparisons was corrected separately.

### Feature Difference Analysis

The effect of perturbation by the presence of CD19-TCB and Teplizumab, respectively, on signaling synapses was analyzed.

First, the images predicted as “synapse w/signaling” were selected. The brightfield (BF) feature was removed as the intensity of BF does not contain biological meaning. Also, the morphological features of BF were already captured based on F-actin masks. Therefore, this information was redundant. Without being bound by this theory, it is assumed that this feature reduction was also necessary as it reduces the number of tests and increases the chance of finding meaningful p-values after correction for multiple testing.

This procedure yielded 210 features for comparison for SEA and TCB based on F-actin, MHCII, CD3, and P-CD3zeta.

For Teplizumab the features were reduced even more. Without being bound by this theory, this reduction was required because of the usage of CD4 in recording images for Teplizumab instead of CD3 used for CD19TCB. Therefore, a meaningful comparison between Teplizumab and its control based on CD3 was not feasible. Thus, 132 features extracted from F-actin, MHCII, and P-CD3zeta were analyzed.

After the feature selection, the features were compared using the Mann-Whitney U test for each condition and its control.

To understand the direction of change, the difference in median of features for each condition and its control was used. To account for multiple testing, the Benjamini-Hochberg procedure with α=0.05 was used. As the conditions were independent, the p-values for each condition and its control were corrected separately.

### Granzyme B Prediction and Feature Ranking

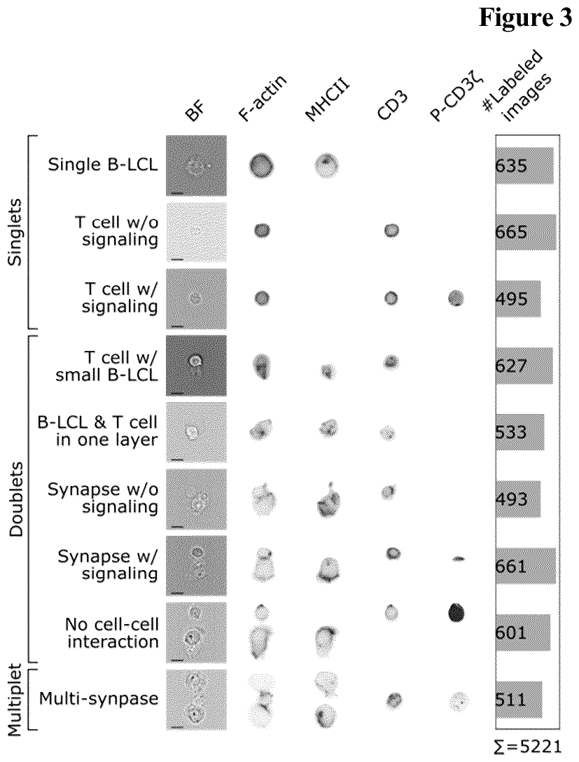

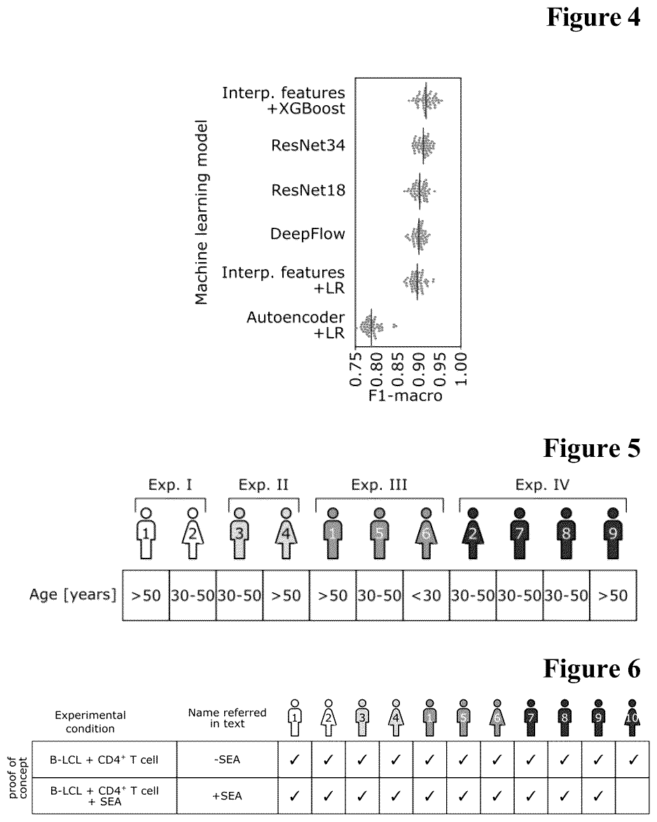

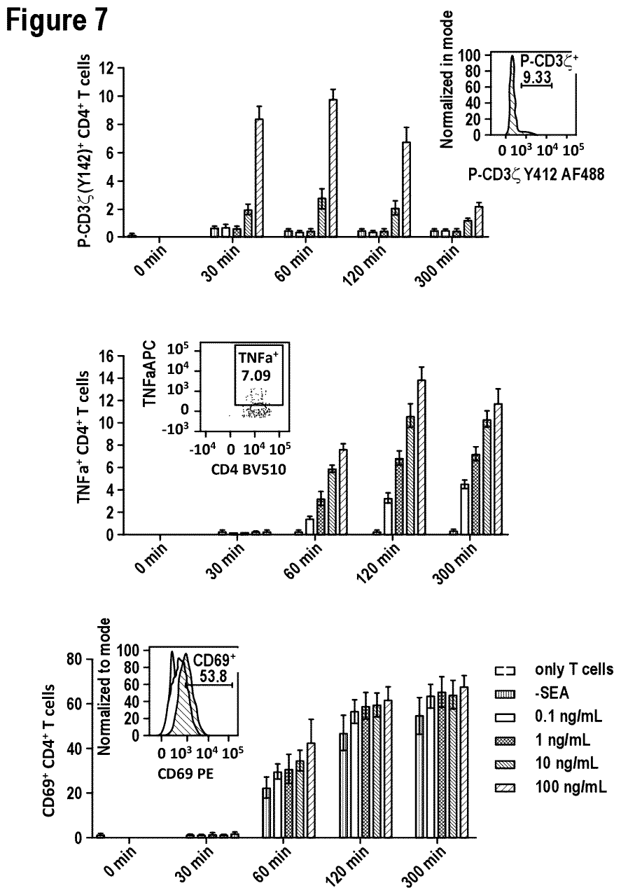

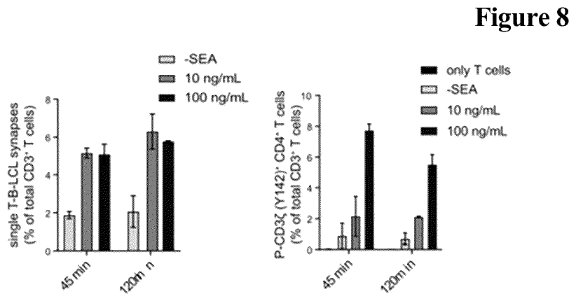

To predict Granzyme B, images predicted as “synapse w/o signaling” and “synapse w/signaling” for each condition were used. Without being bound by this theory, it is assumed that the synapses will lead to cytokine production. Considering that for each donor and condition thousands of images were available. An aggregation pipeline was used to create a feature vector corresponding to each donor and condition. To reduce the number of features, only the consistent feature changes for the CD19-TCB were used (FIG. 3). For each donor and condition, the features were aggregated using 5th, 50th and 95th percentile to capture the extremes and average of every feature.

After deriving the aggregated features, a one-donor-leave-out cross validation was used to train a linear regression model with LassoLars. The most important features were based on the magnitude of the coefficients.

### Visualizations

For the plots and images, matplotlib (version=3.3.2) and seaborn (0.11.2) in Python were used.

### REFERENCES

1. Huppa, J. B., and Davis, M. M., Nat. Rev. Immunol. 3, 973-983 (2003).
2. Dustin, M. L., Cancer Immunol. Res. 2, 1023-1033 (2014).
3. Kearney, C. J., et al., Crit. Rev. Immunol. 35, 325-347 (2015).
4. Kallikourdis, M., et al., Front. Immunol. 6, 433 (2015).
5. Laura, R. D., et al., Jci Insight 3, e120757 (2018).
6. Schubert, D. A., et al., J. Exp. Med. 209, 335-352 (2012).
7. Calvez, R., et al., Haematologica 96, 1415-1423 (2011).
8. Alan, G. R., et al., J. Clin. Invest. 118, 2427-2437 (2008).
9. Francesca, F. and Baldari, C. T., Pharmacol. Res. 134, 118-133 (2018).
10. Tai, Y., et al., Front. Pharmacol. 9, 642 (2018).
11. Scott, M., S.-T., et al., Ann. N.Y. Acad. Sci. 1183, 123-148 (2010).
12. Wetzel, S. A., et al., J. Immunol. 169, 6092-6101 (2002).
13. Cremasco, F. et al., PLOS One 16, e0241091 (2021).
14. Amita, J. et al., J. Clin. Pharmacol. 46, 10-20 (2006).
15. Lee, J. Y. et al., Nat. Commun. 7, 13354 (2016).
16. Dickopf, S., et al., Comput. Struct. Biotechnol. J. 18, 1221-1227 (2020).
17. Cartwright, A. N. R., et al., Nat. Commun. 5, 5479 (2014).
18. Vyver, A. J. V. D., et al., Mol. Cancer. Ther. 20, 357-366 (2021).
19. Wabnitz, G. H., et al., J. Immunol. Meth. 423, 29-39 (2015).
20. Wabnitz, G. H., et al., Eur. J. Immunol. 41, 3157-3169 (2011).
21. Ahmed, F., et al., J. Immunol. Meth. 347, 79-86 (2009).
22. Lippeveld, M., et al., Cytom. Part A 97, 308-319 (2020).
23. Eulenberg, P., et al., Nat. Commun. 8, 463 (2017).
24. Kranich, J., et al., J. Extracell. Vesicles 9, 1792683 (2020).
25. Chlis, N.-K., et al., Nucleic Acids Res. 48, gkaa926-(2020).
26. Doan, M., et al., Nat. Protoc. 16, 3572-3595 (2021).
27. Hennig, H., et al., Methods, San Diego Calif 112, 201-210 (2017).
28. Eulenberg, P., et al., Biorxiv 081364 (2017) doi: 10.1101/081364.
29. Leclercq, G., et al., J. Immunother. Cancer 10, e003766 (2022).
30. Xu, D., et al., Cell Immunol. 200, 16-26 (2000).
31. Herold, K. C., et al., J. Clin. Invest. 111, 409-418 (2003).
32. Cremasco, F., et al., Plos One 16, e0241091 (2021).
33. Bacac, M., et al., Clin. Cancer Res. 22, 3286-3297 (2016).
35. Wabnitz, G., et al., J. Vis. Exp. (2019) doi: 10.3791/55345.
36. Carpenter, A. E., et al., Genome Biol. 7, R100-R100 (2006).
37. German, Y., et al., Cell Reports 36, 109318 (2021).
38. Perakis, A., et al., Arxiv (2021) doi: 10.1007/978-3-030-87589-3_58.
39. Naghizadeh, A., et al., Plos Comput. Biol. 18, e1009883 (2022).
40. Xiong, W., et al., Mol. Ther. 26, 963-975 (2018).
41. Walt, S. van der, et al., Peerj 2, e453 (2014).
42. Harris, C. R., et al., Nature 585, 357-362 (2020).
43. Virtanen, P., et al., Nat. Methods 17, 261-272 (2020).
44. Adler, J. and Parmryd, I., Cytom. Part A 77A, 733-742 (2010).
45. Haralick, R. M., et al., Ieee Transactions Syst. Man. Cybern. SMC-3, 610-621 (1973).
46. Buitinck, L., et al. Arxiv (2013).
47. Haq, A. U., et al., Ieee Access 7, 151482-151492 (2019).
48. Krishnapuram, B., et al., Proc. 22nd Acm. Sigkdd Int. Conf. Knowl. Discov. Data Min. 785-794 (2016) doi: 10.1145/2939672.2939785.

1. A method for classifying cells in a cell mixture, wherein the mixture comprises T-cells and activated B-cells or antigen-presenting cells, comprising the following steps:
a) applying at least labelled antibodies binding to F-actin, MHCII and CD3 to the cell mixture to obtain a labelled cell mixture, wherein the antibodies are each labelled with a dye, wherein the dyes have different emission wavelengths,
b) acquiring at least one image of the cell mixture, and
c) classifying the cells in the cell mixture to be
i) an isolated cell if the cell is a single cell, is F-Actin positive and
is MHCII positive and CD3 negative, or
is MHCII negative and CD3 positive,
ii) a doublet or multiplet of cells if the cell is an aggregate of two or three or more cells, is F-Actin positive, MHCII positive and CD3 positive.

2. The method according to claim 1, wherein
step a) is: applying at least labelled antibodies binding to F-actin, MHCII, CD3 and P-CD3zeta to the cell mixture, wherein the antibodies are each labelled with a dye, wherein the dyes have different emission wavelengths,
and step c) is: classifying the cells in the cell mixture to be
i) a single B-cell or antigen-presenting cell if the cell is F-Actin positive, MHCII positive, CD3 negative and P-CD3zeta negative,
ii) a single T-cell without signaling if the cell is F-Actin positive, MHCII negative, CD3 positive and P-CD3zeta negative,
iii) a single T-cell with signaling if the cell is F-Actin positive, MHCII negative, CD3 positive and P-CD3zeta positive,
iv) a doublet of a B-cell or antigen-presenting cell and a T-cell forming a synapse without signaling if the doublet is F-Actin positive, MHCII positive, CD3 positive and P-CD3zeta negative,
v) a doublet or multiplet of one or two B-cells or antigen-presenting cells and one T-cell forming a synapse with signaling if the doublet or multiplet is F-Actin positive, MHCII positive, CD3 positive and P-CD3zeta positive.

3. The method according to claim 1, wherein step b) is: acquiring images of the cell mixture using an imaging flow cytometer.

4. The method according to claim 1, wherein the acquired images are images each showing a single cell or an isolated doublet or multiplet.

5. The method according to claim 1, wherein the mixture comprises B-cells or antigen-presenting cells and T-cells at a cellular ratio of about 4:3.

6. The method according to claim 1, wherein the T-cells are CD4 positive memory T-cells.

7. The method according to claim 1, wherein the mixture of cells is centrifuged after the mixing.

8. (canceled)
